(19) United States
(12) Patent Application Publication
(10) Pub. No.: US 2018/0339275 A1
(43) Pub. Date: Nov. 29, 2018

(54) GAS SEPARATION MEMBRANE, GAS SEPARATION MODULE, GAS SEPARATOR, AND GAS SEPARATION METHOD

(71) Applicant: FUJIFILM Corporation, Tokyo (JP)

(72) Inventors: Tetsu KITAMURA, Kanagawa (JP); Sotaro INOMATA, Kanagawa (JP); Masatoshi YUMOTO, Kanagawa (JP); Motoi HARADA, Kanagawa (JP)

(73) Assignee: FUJIFILM Corporation, Tokyo (JP)

(21) Appl. No.: 16/045,771

(22) Filed: Jul. 26, 2018

Related U.S. Application Data
(63) Continuation of application No. PCT/JP2017/004465, filed on Feb. 8, 2017.

(30) Foreign Application Priority Data
Feb. 26, 2016 (JP) 2016-036424

(51) Int. Cl. B01D 71/64; B01D 53/22; B01D 69/10; B01D 69/12; C08G 73/10; C07C 7/144

(52) U.S. Cl. CPC B01D 71/64; B01D 53/228; B01D 69/10; C07C 7/144; C08G 73/1092; C08G 73/1067; B01D 69/12

(57) ABSTRACT

A gas separation membrane includes a gas separation layer containing a polyimide compound and the polyimide compound has a repeating unit represented by Formula (I).

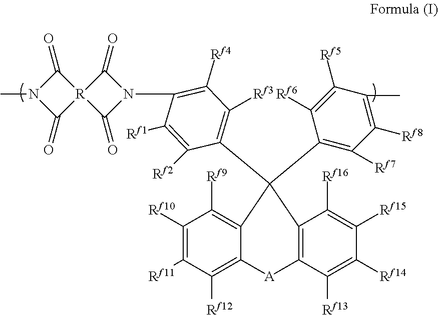

In Formula (I), R^{f1}, R^{f4}, R^{f5}, and R^{f8} each independently represent an alkyl group. R^{f2}, R^{f3}, R^{f6}, R^{f7}, and R^{f9} to R^{f16} each independently represent a hydrogen atom or a substituent, and at least one of R^{f2}, R^{f3}, R^{f6}, R^{f7}, and R^{f9}, . . . , or R^{f16} represents a specific polar group. A represents a single bond or a divalent linking group having a specific structure. R represents a mother nucleus having a specific structure.

### CROSS-REFERENCE TO RELATED APPLICATIONS

This application is a Continuation of PCT International Application No. PCT/JP2017/4465, filed on Feb. 8, 2017, which claims priority under 35 U.S.C. § 119(a) to Japanese Patent Application No. 2016-036424, filed on Feb. 26, 2016. Each of the above application(s) is hereby expressly incorporated by reference, in its entirety, into the present application.

### BACKGROUND OF THE INVENTION

### 1. Field of the Invention

The present invention relates to a gas separation membrane, a gas separation module, a gas separator, and a gas separation method.

### 2. Description of the Related Art

A material formed of a polymer compound has a gas permeability specific to the material. Based on this property, it is possible to cause selective permeation and separation out of a target gas component using a membrane formed of a specific polymer compound. As an industrial application for this gas separation membrane related to the problem of global warming, separation and recovery of carbon dioxide from large-scale carbon dioxide sources using this gas separation membrane has been examined in thermal power plants, cement plants, or ironworks blast furnaces. Further, this membrane separation technique has been attracting attention as means for solving environmental issues which can be performed with relatively little energy. In addition, natural gas or biogas (gas generated due to fermentation or anaerobic digestion, for example, biological excrement, organic fertilizers, biodegradable substances, sewage, garbage, or energy crops) is a mixed gas mainly containing methane and carbon dioxide, and a membrane separation method has been examined as means for removing carbon dioxide and the like which are impurities.

In purification of natural gas using a membrane separation method, excellent gas permeability and gas separation selectivity are required in order to more efficiently separate gas. Various membrane materials have been examined for the purpose of realizing excellent gas permeability and gas separation selectivity, and a gas separation membrane obtained by using a polyimide compound has been examined as part of examination of membrane materials. For example, JP1990-261524A (JP-H02-261524A) describes a polyimide compound obtained by synthesizing a specific site of 4,4′-(9-fluorenylidene)dianiline, to which a specific substituent such as an alkyl group has been introduced, as a diamine monomer and also describes that a membrane formed using this polyimide compound has excellent separation selectivity of oxygen and nitrogen.

### SUMMARY OF THE INVENTION

In order to obtain a practical gas separation membrane, it is necessary to ensure sufficient gas permeability and to realize improved gas separation selectivity. However, gas permeability and gas separation selectivity have a so-called trade-off relationship. Therefore, by adjusting a copolymerization component of a polyimide compound used for a gas separation layer, any of the gas permeability and the gas separation selectivity of the gas separation layer can be improved, but it is considered to be difficult to achieve both properties at high levels.

Further, in an actual plant, a membrane is plasticized due to the influence of impurity components (such as benzene, toluene, and xylene) present in natural gas and this results in a problem of degradation in gas separation selectivity. Accordingly, a gas separation membrane is also required to have plasticity resistance that enables desired gas separation selectivity to be maintained and exhibited in the presence of the impurity components.

However, a polyimide compound typically has degraded plasticity resistance, and the gas separation performance thereof is likely to be degraded in the coexistence of impurity components such as toluene. Particularly in a case where a polyimide compound having a high gas permeability is used for a gas separation layer, the gas separation layer is easily affected by the impurity components, and thus swelling of the gas separation layer is promoted. Therefore, in the gas separation layer obtained by using a polyimide compound, it is difficult to achieve both of the gas permeability and the plasticity resistance at high levels.

The present invention relates to a gas separation membrane which enables gas separation with a high speed and high selectivity by achieving both of excellent gas permeability and excellent gas separation selectivity at high levels even in a case of being used under a high pressure condition and is capable of satisfactorily maintaining gas separation selectivity even in a case of being brought into contact with impurity components such as toluene. Further, the present invention relates to a gas separation module, a gas separator, and a gas separation method obtained by using the gas separation membrane.

As the result of intensive examination repeatedly conducted by the present inventors, they found the following. A polyimide compound is obtained by employing a 4,4′-(9-fluorenylidene)dianiline skeleton as a diamine component of a polyimide compound, introducing an alkyl group to each specific position in two benzene rings having a linking site to be incorporated to a polyimide main chain of this skeleton, and introducing a specific polar group to at least one benzene ring from among four benzene rings constituting this skeleton. In a case where such a polyimide compound is used for a gas separation layer of a gas separation membrane, this gas separation membrane exhibits excellent gas permeability, is unlikely to be affected by impurity components such as toluene due to excellent gas separation selectivity, and exhibits excellent plasticity resistance. The present invention has been completed after repeated examination based on these findings.

The present invention includes the following aspects.

[1] A gas separation membrane comprising: a gas separation layer which contains a polyimide compound, in which the polyimide compound has a repeating unit represented by Formula (I),

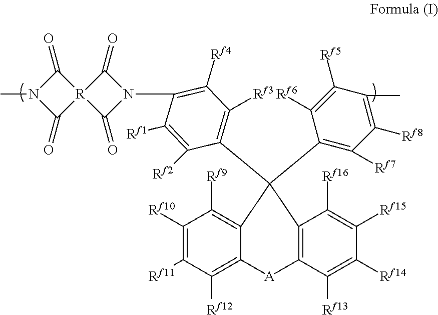

in Formula (I), A represents a divalent linking group selected from a single bond, —CR^{L1}CR^{L2}—, —O—, —S—, and —NR^{L3}—, R^{L1}, R^{L2}, and R^{L3} each independently represent a hydrogen atom or a substituent,

R^{f1}, R^{f4}, R^{f5}, and R^{f8} each independently represent an alkyl group,

R^{f2}, R^{f3}, R^{f6}, R^{f7}, and R^{f9} to R^{f16} each independently represent a hydrogen atom or a substituent,

provided that at least one of R^{f2}, R^{f3}, R^{f6}, R^{f7}, and R^{f9}, . . . , or R^{f16} represents a polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom, and

R represents a tetravalent group represented by any of Formulae (I-1) to (I-28), where X¹ to X³ each independently represent a single bond or a divalent linking group, L represents —CH═CH— or —CH₂—, R¹ and R² each independently represent a hydrogen atom or a substituent, and the symbol “*” represents a bonding site with respect to a carbonyl group in Formula (I).

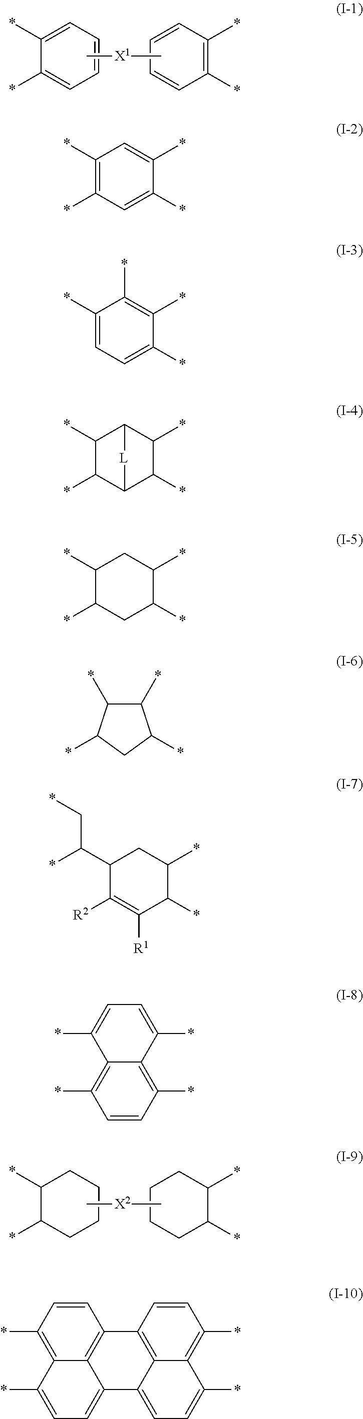

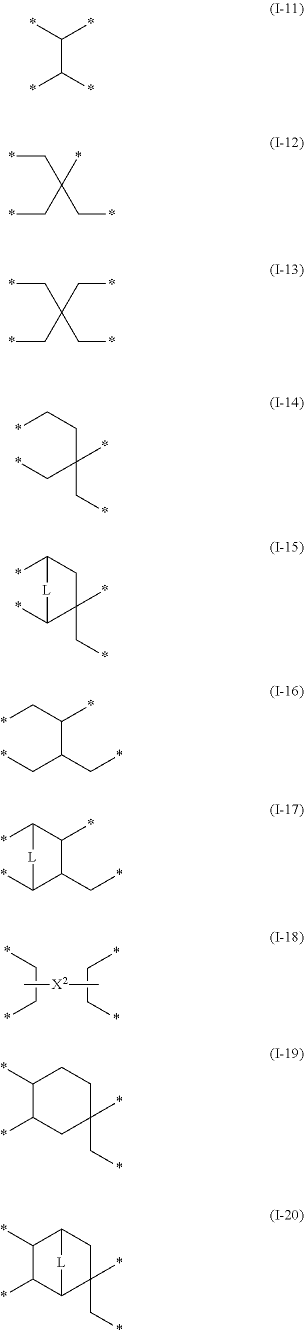

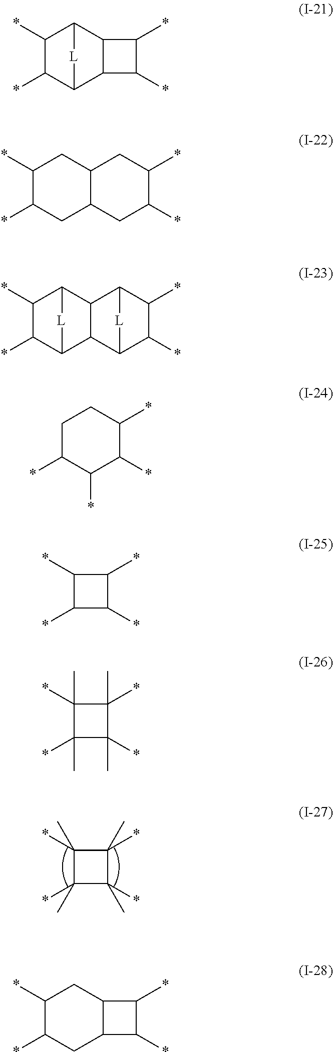

[2] The gas separation membrane according to [1], in which A in Formula (I) represents a single bond.

[3] The gas separation membrane according to [1] or [2], in which R^{f10} and/or R^{f15} in Formula (I) represents a polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom.

[4] The gas separation membrane according to any one of [1] to [3], in which any two to four of R^{f2}, R^{f3}, R^{f6}, R^{f7}, and R^{f9} to R^{f16} in Formula (I) represent a polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom.

[5] The gas separation membrane according to any one of [1] to [4], in which at least one of R^{f2}, R^{f3}, R^{f6}, R^{f7}, and R^{f9}, . . . , or R^{f16} in Formula (I) represents a sulfamoyl group.

[6] The gas separation membrane according to any one of [1] to [5], in which R^{f1}, R^{f4}, R^{f5}, and R^{f8} in Formula (I) represent methyl.

[7] The gas separation membrane according to any one of [1] to [6], in which the repeating unit represented by Formula (I) is represented by Formula (I-a),

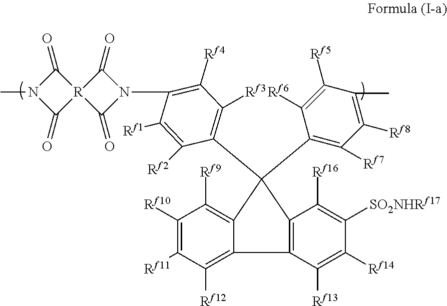

in Formula (I-a), R^{f1} to R^{f14}, R^{f16}, and R each have the same definition as that for R^{f1} to R^{f14}, R^{f16}, and R in Formula (I), and R^{f17} represents a hydrogen atom or a substituent.

[8] The gas separation membrane according to any one of [1] to [7], in which the polyimide compound further has at least one repeating unit selected from a repeating unit represented by Formula (II-a) and a repeating unit represented by Formula (II-b),

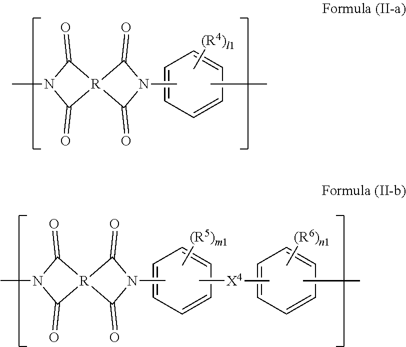

in Formulae (II-a) and (II-b), R has the same definition as that for R in Formula (I), R⁴ to R⁶ each independently represent a substituent, l1, m1, and n1 each independently represent an integer of 0 to 4, and X⁴ represents a single bond or a divalent linking group, provided that the repeating unit represented by Formula (II-b) does not include the repeating unit included in the repeating unit represented by Formula (I).

[9] The gas separation membrane according to [8], in which a ratio of a molar amount of the repeating unit represented by Formula (I) to a total molar amount of the repeating unit represented by Formula (I), the repeating unit represented by Formula (II-a), and the repeating unit represented by Formula (II-b) in the polyimide compound is 50% by mole or greater and less than 100% by mole.

[10] The gas separation membrane according to [8] or [9], in which the polyimide compound is formed of the repeating unit represented by Formula (I) and the repeating unit represented by Formula (II-a), the repeating unit represented by Formula (I) and the repeating unit represented by Formula (II-b), or the repeating unit represented by Formula (I), the repeating unit represented by Formula (II-a) and the repeating unit represented by Formula (II-b).

[11] The gas separation membrane according to any one of [1] to [7], in which the polyimide compound does not have any of a repeating unit represented by Formula (II-a) and a repeating unit represented by Formula (II-b),

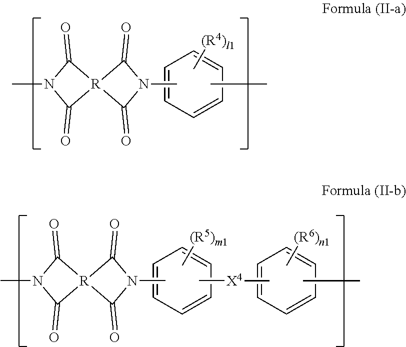

in Formulae (II-a) and (II-b), R has the same definition as that for R in Formula (I), R⁴ to R⁶ each independently represent a substituent, l1, m1, and n1 each independently represent an integer of 0 to 4, and X⁴ represents a single bond or a divalent linking group, provided that the repeating unit represented by Formula (II-b) does not include the repeating unit included in the repeating unit represented by Formula (I).

[12] The gas separation membrane according to [11], in which the polyimide compound is formed of the repeating unit represented by Formula (I).

[13] The gas separation membrane according to any one of [1] to [12], in which the gas separation membrane further comprises a gas permeating support layer and is a gas separation composite membrane in which the gas separation layer is provided on the upper side of the gas permeating support layer.

[14] The gas separation membrane according to [13], in which the gas permeating support layer includes a porous layer and a non-woven fabric layer, and the gas separation layer, the porous layer, and the non-woven fabric layer are provided in this order.

[15] The gas separation membrane according to any one of [1] to [14], in which a permeation rate of carbon dioxide in a mixed gas containing carbon dioxide and methane at 40° C. and 5 MPa is greater than 20 GPU, and a ratio (R_{CO2}/R_{CH4}) between permeation rates of the carbon dioxide and the methane is 15 or greater.

[16] The gas separation membrane according to any one of [1] to [15], which is used for selective permeation of carbon dioxide from the mixed gas containing carbon dioxide and methane.

[17] A gas separation module comprising: the gas separation membrane according to any one of [1] to [16].

[18] A gas separator comprising: the gas separation module according to [17].

[19] A gas separation method which is performed by using the gas separation membrane according to any one of [1] to [16].

The numerical ranges shown using “to” in the present specification indicate ranges including the numerical values described before and after “to” as the lower limits and the upper limits.

In the present specification, in a case where a plurality of substituents or linking groups (hereinafter, referred to as substituents or the like) shown by specific symbols are present or a plurality of substituents are defined simultaneously or alternatively, this means that the respective substituents may be the same as or different from each other. The same applies to the definition of the number of substituents or the like. Moreover, in a case where there is a repetition of a plurality of partial structures shown by means of the same display in the formula, the respective partial structures or repeating units may be the same as or different from each other.

In regard to compounds or groups described in the present specification, the description includes salts thereof and ions thereof in addition to the compounds or the groups. Further, the description includes those obtained by changing a part of the structure of the compounds or the groups within the range in which the effects of the purpose are exhibited.

A substituent or a linking group in which substitution or unsubstitution is not specified in the present specification may include an optional substituent of the group within a range in which desired effects are exhibited. The same applies to a compound in which substitution or unsubstitution is not specified.

A preferable range of a substituent group Z described below is set as a preferable range of a substituent in the present specification unless otherwise specified.

The gas separation membrane, the gas separation module, and the gas separator of the present invention enable achievement both of excellent gas permeability and excellent gas separation selectivity at high levels and enable gas separation with a high speed and high selectivity even in a case of being used under a high pressure condition.

According to the gas separation method of the present invention, it is possible to separate gas with excellent gas permeability and excellent gas separation selectivity and to perform gas separation with a high speed and high selectivity even under a high pressure condition.

### BRIEF DESCRIPTION OF THE DRAWINGS

FIG. 1 is a cross-sectional view schematically illustrating an embodiment of a gas separation composite membrane according to the present invention.

FIG. 2 is a cross-sectional view schematically illustrating another embodiment of a gas separation composite membrane according to the present invention.

### DESCRIPTION OF THE PREFERRED EMBODIMENTS

Hereinafter, preferred embodiments of the present invention will be described.

A gas separation membrane of the present invention contains a specific polyimide compound in a gas separation layer.

[Polyimide Compound]

The polyimide compound used in the present invention has a repeating unit represented by Formula (I).

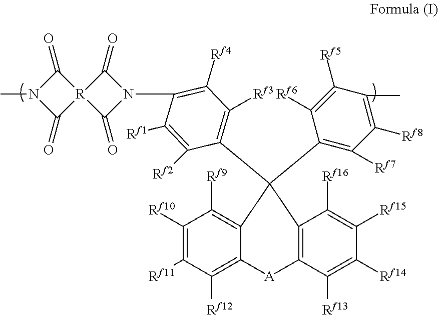

In Formula (I), A represents a divalent linking group selected from a single bond, —CR^{L1}CR^{L2}—, —O—, —S—, and —NR^{L3}—.

R^{L1}, R^{L2}, and R^{L3} each independently represent a hydrogen atom or a substituent. Examples of the substituent which can be employed as R^{L1}, R^{L2}, and R^{L3} include groups selected from the following substituent group Z. Among these, an alkyl group or an aryl group is preferable.

It is more preferable that A represents a single bond.

R^{f1}, R^{f4}, R^{f5}, and R^{f8} each independently represent an alkyl group. The alkyl group may be linear, branched, or cyclic. The number of carbon atoms of the alkyl group is preferably in a range of 1 to 10, more preferably in a range of 1 to 6, and still more preferably in a range of 1 to 3. Further, it is also preferable that the alkyl group contains a fluorine atom as a substituent. R^{f1}, R^{f4}, R^{f5}, and R^{f8} each independently represent more preferably methyl, trifluoromethyl, or ethyl and particularly preferably methyl.

R^{f2}, R^{f3}, R^{f6}, R^{f7}, and R^{f9} to R^{f16} each independently represent a hydrogen atom or a substituent. Examples of the substituent which can be employed as R^{f2}, R^{f3}, R^{f6}, R^{f7}, and R^{f9} to R^{f16} include groups selected from the following substituent group Z. Here, at least one of R^{f2}, R^{f3}, R^{f6}, R^{f7}, and R^{f9}, . . . , or R^{f16} represents a polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom. Hereinafter, the polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom is referred to as a “polar group T”.

In a case where the polar group T is the sulfamoyl group, the sulfamoyl group may be unsubstituted or include a substituent. In a case where the sulfamoyl group includes a substituent, examples of such a substituent include groups selected from the following substituent group Z. Among these, an alkyl group (preferably an alkyl group having 1 to 10 carbon atoms, more preferably an alkyl group having 1 to 6 carbon atoms, still more preferably an alkyl group having 1 to 3 carbon atoms, and even still more preferably methyl or ethyl), a cycloalkyl group (preferably a cycloalkyl group having 3 to 18 carbon atoms, more preferably a cycloalkyl group having 4 to 12 carbon atoms, and still more preferably a cycloalkyl group having 5 to 10 carbon atoms), or an aryl group (preferably an aryl group having 6 to 20 carbon atoms, more preferably an aryl group having 6 to 15 carbon atoms, still more preferably an aryl group having 6 to 12 carbon atoms, and even still more preferably a phenyl group) is preferable.

It is particularly preferable that the sulfamoyl group which can be employed as the polar group T is unsubstituted.

In a case where the polar group T is the carbamoyl group, the carbamoyl group may be unsubstituted or include a substituent. In a case where the carbamoyl group includes a substituent, examples of such a substituent include groups selected from the following substituent group Z. Among these, an alkyl group (preferably an alkyl group having 1 to 10 carbon atoms, more preferably an alkyl group having 1 to 6 carbon atoms, still more preferably an alkyl group having 1 to 3 carbon atoms, and even still more preferably methyl or ethyl), a cycloalkyl group (preferably a cycloalkyl group having 3 to 18 carbon atoms, more preferably a cycloalkyl group having 4 to 12 carbon atoms, and still more preferably a cycloalkyl group having 5 to 10 carbon atoms), or an aryl group (preferably an aryl group having 6 to 20 carbon atoms, more preferably an aryl group having 6 to 15 carbon atoms, still more preferably an aryl group having 6 to 12 carbon atoms, and even still more preferably a phenyl group) is preferable.

It is particularly preferable that the carbamoyl group which can be employed as the polar group T is unsubstituted.

In a case where the polar group T is the acyloxy group, the number of carbon atoms thereof is preferably in a range of 2 to 20 and more preferably in a range of 2 to 10. Examples of the acyloxy group include acetoxy and benzoyloxy.

In a case where the polar group T is the alkoxycarbonyl group, the number of carbon atoms thereof is preferably in a range of 2 to 20 and more preferably in a range of 2 to 10. Examples of the alkoxycarbonyl group include methoxycarbonyl and ethoxycarbonyl.

In a case where the polar group T is the aryloxycarbonyl group, the number of carbon atoms thereof is preferably in a range of 7 to 20 and more preferably in a range of 7 to 10. Examples of the aryloxycarbonyl group include phenoxycarbonyl.

Examples of the halogen atom which can be employed as the polar group T include a fluorine atom, a chlorine atom, a bromine atom, and an iodine atom. Among these, a fluorine atom is preferable.

In a case where any of R^{f2}, R^{f3}, R^{f6}, R^{f7}, and R^{f9} to R^{f16} represent a substituent other than the polar group T, an alkyl group, an alkoxy group, or a hydrogen atom is preferable as such a substituent.

In a case where any of R^{f2}, R^{f3}, R^{f6}, R^{f7}, and R^{f9} to R^{f16} represent an alkyl group, the alkyl group may be linear or branched. The number of carbon atoms of the alkyl group is preferably in a range of 1 to 10, more preferably in a range of 1 to 6, and still more preferably in a range of 1 to 3. Further, methyl, trifluoromethyl, or ethyl is even still more preferable as the alkyl group.

In a case where any of R^{f2}, R^{f3}, R^{f6}, R^{f7}, and R^{f9} to R^{f16} represent an alkoxy group, the alkoxy group may be linear or branched. The number of carbon atoms of the alkoxy group is preferably in a range of 1 to 10, more preferably in a range of 1 to 6, and still more preferably in a range of 1 to 3. Further, methoxy or ethoxy is even still more preferable as the alkoxy group.

In a case where only one of R^{f2}, R^{f3}, R^{f6}, R^{f7}, and R^{f9} to R^{f16} represents the polar group T, as such a polar group T, a sulfamoyl group or a carboxy group is preferable, and a sulfamoyl group is more preferable.

In a case where two or more of R^{f2}, R^{f3}, R^{f6}, R^{f7}, and R^{f9} to R^{f16} represent the polar group T, it is preferable that at least one of these two or more polar groups T is a sulfamoyl group or a carboxy group and more preferable that at least one of these two or more polar groups T is a sulfamoyl group.

In the case where two or more of R^{f2}, R^{f3}, R^{f6}, R^{f7}, and R^{f9} to R^{f16} represent the polar group T, it is preferable that all of these two or more polar groups T are groups selected from a sulfamoyl group and a carboxy group and more preferable that all of these two or more polar groups T are sulfamoyl groups.

In Formula (I), it is preferable that R^{f10} and/or R^{f15} represents the polar group T.

It is preferable that any one to four of R^{f2}, R^{f3}, R^{f6}, R^{f7}, and R^{f9} to R^{f16} represent the polar group T and also preferable that any two to four of R^{f2}, R^{f3}, R^{f6}, R^{f7}, and R^{f9} to R^{f16} represent the polar group T. It is particularly preferable that any one or two of R^{f2}, R^{f3}, R^{f6}, R^{f7}, and R^{f9} to R^{f16} represent the polar group T. From the viewpoint of improving the gas permeability, it is preferable that the number of polar groups T as R^{f2}, R^{f3}, R^{f6}, R^{f7}, and R^{f9} to R^{f16} is one.

In a case where any of R^{f2}, R^{f3}, R^{f6}, R^{f7}, and R^{f9} to R^{f16} represent the polar group T, it is particularly preferable that the rest represent a hydrogen atom.

According to the present invention, in the polyimide compound used in the gas separation layer, the diamine component has a 4,4′-(9-fluorenylidene)dianiline skeleton as described above. This skeleton has a twisted structure and voids are likely to be generated in the gas separation layer due to such a structure. Accordingly, in a case where the polyimide compound having a 4,4′-(9-fluorenylidene)dianiline skeleton is applied to the gas separation layer, this application is relatively advantageous in terms that the gas permeability is improved, but the gas separation selectivity is deteriorated. However, the present inventors found that the gas separation selectivity is improved and the plasticity resistance can be improved while the gas permeability is further improved by introducing the substituent defined in Formula (I) into the 4,4′-(9-fluorenylidene)dianiline skeleton. The reason for this is not clear, but can be assumed as follows.

In other words, in the polyimide compound having a repeating unit represented by Formula (I), since alkyl groups (that is, all of R^{f1}, R^{f4}, R^{f5}, and R^{f8} represent an alkyl group) are present so as to interpose a linking site for being incorporated in a polyimide main chain of the 4,4′-(9-fluorenylidene)dianiline skeleton, moderate steric hindrance occurs. Further, since the interaction of the polar group T works, the polyimide compound is moderately densified while maintaining the voids. It is considered that the permeability of molecules with a large dynamic molecular diameter can be effectively suppressed and the permeability of molecules with a small dynamic molecular diameter can be improved due to these complex factors. Further, it is considered that swelling of the membrane occurring at the time of being brought into contact with impurity components such as toluene is effectively suppressed and the plasticity resistance is improved due to the densification of the polyimide compound caused by the interaction of the polar group T.

In Formula (I), R represents a group having a structure represented by any of Formulae (I-1) to (I-28). Here, X¹ to X³ each independently represent a single bond or a divalent linking group, L represents —CH═CH— or —CH₂—, R¹ and R² each independently represent a hydrogen atom or a substituent, and the symbol “*” represents a bonding site with respect to a carbonyl group in Formula (I). R represents preferably a group represented by Formula (I-1), (I-2), or (I-4), more preferably a group represented by Formula (I-1) or (I-4), and particularly preferably a group represented by Formula (I-1).

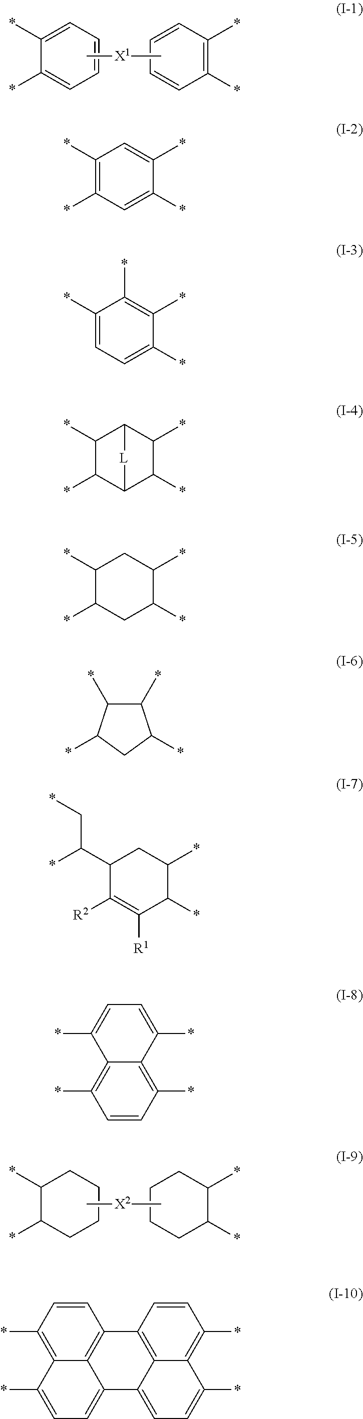

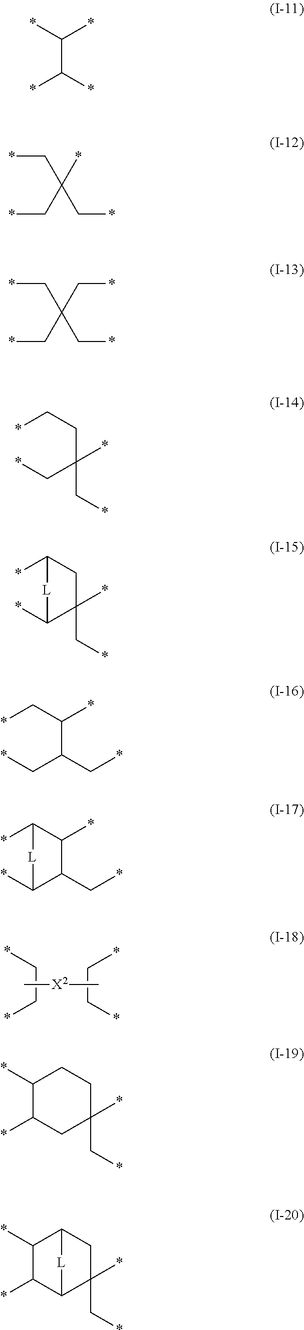

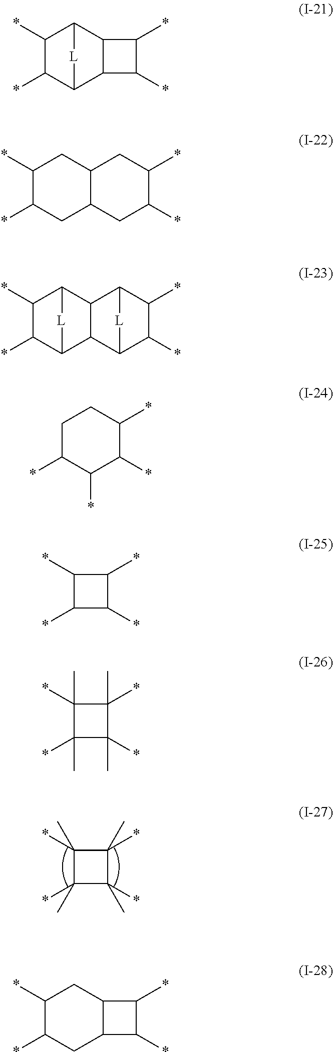

In Formulae (I-1), (I-9), and (I-18), X¹ to X³ each independently represent a single bond or a divalent linking group. As the divalent linking group, —C(R^{x})₂— (R^{x} represents a hydrogen atom or a substituent, and in a case where R^{x} represents a substituent, R^{x}'s may be linked to each other to form a ring), —O—, —SO₂—, —C(═O)—, —S—, —NR^{Y}— (R^{Y} represents a hydrogen atom, an alkyl group (preferably a methyl group or an ethyl group), an aryl group (preferably a phenyl group)), —C₆H₄— (a phenylene group), or a combination of these is preferable, and a single bond or —C(R^{x})₂— is more preferable. In a case where R^{x} represents a substituent, specific examples thereof include groups selected from a substituent group Z described below. Among these, an alkyl group (the preferable range is the same as that of the alkyl group in the substituent group Z described below) is preferable, an alkyl group having a halogen atom as a substituent is more preferable, and trifluoromethyl is particularly preferable. Moreover, in Formula (I-18), X³ is linked to any one of two carbon atoms shown on the left side thereof and any one of two carbon atoms shown on the right side thereof.

X¹ to X³ have a molecular weight of preferably 0 to 300 (the molecular weight is 0 in a case of representing a single bond) and more preferably 0 to 160.

In Formulae (I-4), (I-15), (I-17), (I-20), (I-21), and (I-23), L represents —CH═CH— or —CH₂—.

In Formula (I-7), R¹ and R² each independently represent a hydrogen atom or a substituent. Examples of such a substituent include groups selected from the substituent group Z described below. R¹ and R² may be bonded to each other to form a ring.

R¹ and R² each independently represent preferably a hydrogen atom or an alkyl group, more preferably a hydrogen atom, a methyl group, or an ethyl group, and still more preferably a hydrogen atom.

A substituent may be further added to the carbon atom shown in Formulae (I-1) to (I-28). Specific examples of the substituent include groups selected from the substituent group Z described below. Among these, an alkyl group or an aryl group is preferable.

It is preferable that the repeating unit represented by Formula (I) is represented by Formula (I-a).

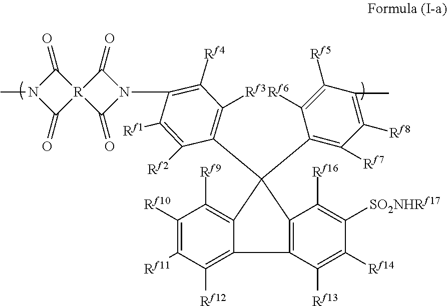

In Formula (I-a), R^{f1} to R^{f14}, R^{f16}, and R each have the same definition as that for R^{f1} to R^{f14}, R^{f16}, and R in Formula (I), and the preferred forms thereof are the same as described above.

In Formula (I-a), it is preferable that R^{f10} represents a hydrogen atom or the polar group (preferably a sulfamoyl group or a carboxy group).

Further, in a case where any of R^{f2}, R^{f3}, R^{f6}, R^{f7}, R^{f9} to R^{f14}, and R^{f16} represent a group other than the polar group T, it is preferable that any of R^{f2}, R^{f3}, R^{f6}, R^{f7}, R^{f9} to R^{f14}, and R^{f16} represent a hydrogen atom.

In Formula (I-a), it is preferable that any one to three of R^{f2}, R^{f3}, R^{f6}, R^{f7}, R^{f9} to R^{f14} and R^{f16} represent the polar group T and also preferable that any two or three of R^{f2}, R^{f3}, R^{f6}, R^{f7}, R^{f9} to R^{f14}, and R^{f16} represent the polar group T. It is particularly preferable that none or any one of R^{f2}, R^{f3}, R^{f6}, R^{f7}, R^{f9} to R^{f14}, and R^{f16} represents the polar group T. From the viewpoint of improving the gas permeability, it is preferable that the number of polar groups as R^{f2}, R^{f3}, R^{f6}, R^{f7}, R^{f9}, to R^{f14}, and R^{f16}, is zero.

R^{f17} represents a hydrogen atom or a substituent. Examples of the substituent which can be employed as R^{f17} include groups selected from the following substituent group Z. Among these, an alkyl group (preferably an alkyl group having 1 to 10 carbon atoms, more preferably an alkyl group having 1 to 6 carbon atoms, still more preferably an alkyl group having 1 to 3 carbon atoms, and even still more preferably methyl or ethyl), a cycloalkyl group (preferably a cycloalkyl group having 3 to 18 carbon atoms, more preferably a cycloalkyl group having 4 to 12 carbon atoms, and still more preferably a cycloalkyl group having 5 to 10 carbon atoms), or an aryl group (preferably an aryl group having 6 to 20 carbon atoms, more preferably an aryl group having 6 to 15 carbon atoms, still more preferably an aryl group having 6 to 12 carbon atoms, and even still more preferably a phenyl group) is preferable.

It is more preferable that R^{F17} represents a hydrogen atom.

The polyimide compound may have a repeating unit represented by Formula (II-a) or a repeating unit represented by Formula (II-b) in addition to the repeating unit represented by Formula (I). Here, the repeating unit represented by Formula (II-b) does not include the repeating unit included in the repeating unit represented by Formula (I).

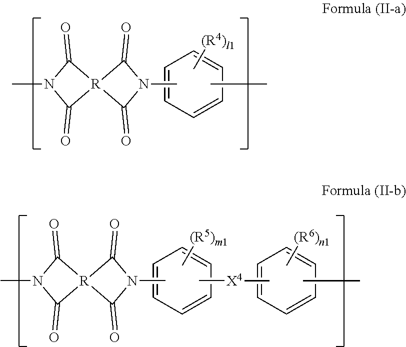

In Formulae (II-a) and (II-b), R has the same definition as that for R in Formula (I) and the preferred forms are the same as each other. R⁴ to R⁶ each independently represent a substituent. Examples of the substituent include groups selected from the substituent group Z described below.

It is preferable that R⁴ represents an alkyl group, a carboxy group, or a halogen atom. l1 showing the number of R⁴'s represents an integer of 0 to 4. In a case where R⁴ represents an alkyl group, l1 represents preferably 1 to 4, more preferably 2 to 4, and still more preferably 3 or 4. In a case where R⁴ represents a carboxy group, l1 represents preferably 1 or 2 and more preferably 1. In a case where R⁴ represents alkyl, the number of carbon atoms in alkyl groups is preferably in a range of 1 to 10, more preferably in a range of 1 to 5, and still more preferably in a range of 1 to 3. It is even still more preferable that the alkyl group is methyl, ethyl, or trifluoromethyl.

In Formula (II-a), it is preferable that both of two linking sites for being incorporated in the polyimide compound of the diamine component (that is, a phenylene group which can contain R⁴) are positioned in the meta position or the para position and more preferable that both of two linking sites are positioned in the para position.

In addition, the structure represented by formula (II-a) does not include the structure represented by Formula (I).

It is preferable that R⁵ and R⁶ each independently represent an alkyl group or a halogen atom or represent a group that forms a ring together with X⁴ by being linked to each other. Further, the form of two R⁵'s being linked to each other to form a ring or the form of two R⁶'s being linked to each other to form a ring is preferable. The structure formed by R⁵ and R⁶ being linked to each other is not particularly limited, but a single bond, —O—, or —S— is preferable. m1 showing the number of R⁵'s and n1 showing the number of R⁶'s each independently represent an integer of 0 to 4, preferably in a range of 1 to 4, more preferably in a range of 2 to 4, and still more preferably 3 or 4. In a case where R⁵ and R⁶ each independently represent an alkyl group, the number of carbon atoms in the alkyl group is preferably in a range of 1 to 10, more preferably in a range of 1 to 5, and still more preferably in a range of 1 to 3. It is even still more preferable that the alkyl group is methyl, ethyl, or trifluoromethyl.

In Formula (II-b), it is preferable that two linking sites for being incorporated in the polyimide compound of two phenylene groups (that is, two phenylene groups which can contain R⁵ and R⁶) in the diamine component are positioned in the meta position or the para position with respect to the linking site of X⁴.

X⁴ has the same definition as that for X¹ in Formula (I-1) and the preferred forms are the same as each other.

In the structure of the polyimide compound, the ratio of the molar amount of the repeating unit represented by Formula (I) to the total molar amount of the repeating unit represented by Formula (I), the repeating unit represented by Formula (II-a), and the repeating unit represented by Formula (II-b) is preferably in a range of 50% to 100% by mole, more preferably in a range of 70% to 100% by mole, still more preferably in a range of 80% to 100% by mole, and even still more preferably in a range of 90% to 100% by mole. Further, the expression “the ratio of the molar amount of the repeating unit represented by Formula (I) to the total molar amount of the repeating unit represented by Formula (I), the repeating unit represented by Formula (II-a), and the repeating unit represented by Formula (II-b) is 100% by mole” means that the polyimide compound does not have any of the repeating unit represented by Formula (II-a) or the repeating unit represented by Formula (II-b).

In a case where the polyimide compound has the repeating unit represented by Formula (I) or a repeating unit other than the repeating unit represented by Formula (I), it is preferable that the remainder other than the repeating unit represented by Formula (I) is formed of the repeating unit represented by Formula (II-a) or the repeating unit represented by Formula (II-b). Here, the concept “formed of the repeating unit represented by Formula (II-a) or the repeating unit represented by Formula (II-b)” includes three forms, which are, a form formed of the repeating unit represented by Formula (II-a), a form formed of the repeating unit represented by Formula (II-b), and a form formed of the repeating unit represented by Formula (II-a) and the repeating unit represented by Formula (II-b). In other words, it is preferable that the polyimide compound is formed of the repeating unit represented by Formula (I), the repeating unit represented by Formula (I) and the repeating unit represented by Formula (II-a), the repeating unit represented by Formula (I) and the repeating unit represented by Formula (II-b), or the repeating unit represented by Formula (I), the repeating unit represented by Formula (II-a), and the repeating unit represented by Formula (II-b).

Examples of the substituent group Z include:

an alkyl group (the number of carbon atoms of the alkyl group is preferably in a range of 1 to 30, more preferably in a range of 1 to 20, and particularly preferably in a range of 1 to 10, and examples thereof include methyl, ethyl, iso-propyl, tert-butyl, n-octyl, n-decyl, and n-hexadecyl), a cycloalkyl group (the number of carbon atoms of the cycloalkyl group is preferably in a range of 3 to 30, more preferably in a range of 3 to 20, and particularly preferably in a range of 3 to 10, and examples thereof include cyclopropyl, cyclopentyl, and cyclohexyl), an alkenyl group (the number of carbon atoms of the alkenyl group is preferably in a range of 2 to 30, more preferably in a range of 2 to 20, and particularly preferably in a range of 2 to 10, and examples thereof include vinyl, allyl, 2-butenyl, and 3-pentenyl), an alkynyl group (the number of carbon atoms of the alkynyl group is preferably in a range of 2 to 30, more preferably in a range of 2 to 20, and particularly preferably in a range of 2 to 10, and examples thereof include propargyl and 3-pentynyl), an aryl group (the number of carbon atoms of the aryl group is preferably in a range of 6 to 30, more preferably in a range of 6 to 20, and particularly preferably in a range of 6 to 12, and examples thereof include phenyl, p-methylphenyl, naphthyl, and anthranyl), an amino group (such as an amino group, an alkylamino group, an arylamino group, or a heterocyclic amino group; the number of carbon atoms of the amino group is preferably in a range of 0 to 30, more preferably in a range of 0 to 20, and particularly preferably in a range of 0 to 10 and examples thereof include amino, methylamino, dimethylamino, diethylamino, dibenzylamino, diphenylamino, and ditolylamino), an alkoxy group (the number of carbon atoms of the alkoxy group is preferably in a range of 1 to 30, more preferably in a range of 1 to 20, and particularly preferably in a range of 1 to 10, and examples thereof include methoxy, ethoxy, butoxy, and 2-ethylhexyloxy), an aryloxy group (the number of carbon atoms of the aryloxy group is preferably in a range of 6 to 30, more preferably in a range of 6 to 20, and particularly preferably in a range of 6 to 12, and examples thereof include phenyloxy, 1-naphthyloxy, and 2-naphthyloxy), a heterocyclic oxy group (the number of carbon atoms of the heterocyclic oxy group is preferably in a range of 1 to 30, more preferably in a range of 1 to 20, and particularly preferably in a range of 1 to 12, and examples thereof include pyridyloxy, pyrazyloxy, pyrimidyloxy, and quinolyloxy),

an acyl group (the number of carbon atoms of the acyl group is preferably in a range of 1 to 30, more preferably in a range of 1 to 20, and particularly preferably in a range of 1 to 12, and examples thereof include acetyl, benzoyl, formyl, and pivaloyl), an alkoxycarbonyl group (the number of carbon atoms of the alkoxycarbonyl group is preferably in a range of 2 to 30, more preferably in a range of 2 to 20, and particularly preferably in a range of 2 to 12, and examples thereof include methoxycarbonyl and ethoxycarbonyl), an aryloxycarbonyl group (the number of carbon atoms of the aryloxycarbonyl group is preferably in a range of 7 to 30, more preferably in a range of 7 to 20, and particularly preferably in a range of 7 to 12, and examples thereof include phenyloxycarbonyl), an acyloxy group (the number of carbon atoms of the acyloxy group is preferably in a range of 2 to 30, more preferably in a range of 2 to 20, and particularly preferably in a range of 2 to 10, and examples thereof include acetoxy and benzoyloxy), an acylamino group (the number of carbon atoms of the acylamino group is preferably in a range of 2 to 30, more preferably in a range of 2 to 20, and particularly preferably in a range of 2 to 10, and examples thereof include acetylamino and benzoylamino),

an alkoxycarbonylamino group (the number of carbon atoms of the alkoxycarbonylamino group is preferably in a range of 2 to 30, more preferably in a range of 2 to 20, and particularly preferably in a range of 2 to 12, and examples thereof include methoxycarbonylamino), an aryloxycarbonylamino group (the number of carbon atoms of the aryloxycarbonylamino group is preferably in a range of 7 to 30, more preferably in a range of 7 to 20, and particularly preferably in a range of 7 to 12, and examples thereof include phenyloxycarbonylamino), a sulfonylamino group (the number of carbon atoms of the sulfonylamino group is preferably in a range of 1 to 30, more preferably in a range of 1 to 20, and particularly preferably in a range of 1 to 12, and examples thereof include methanesulfonylamino and benzenesulfonylamino), a sulfamoyl group (the number of carbon atoms of the sulfamoyl group is preferably in a range of 0 to 30, more preferably in a range of 0 to 20, and particularly preferably in a range of 0 to 12, and examples thereof include sulfamoyl, methylsulfamoyl, dimethylsulfamoyl, and phenylsulfamoyl),

an alkylthio group (the number of carbon atoms of the alkylthio group is preferably in a range of 1 to 30, more preferably in a range of 1 to 20, and particularly preferably in a range of 1 to 12, and examples thereof include methylthio and ethylthio), an arylthio group (the number of carbon atoms of the arylthio group is preferably in a range of 6 to 30, more preferably in a range of 6 to 20, and particularly preferably in a range of 6 to 12, and examples thereof include phenylthio), a heterocyclic thio group (the number of carbon atoms of the heterocyclic thio group is preferably in a range of 1 to 30, more preferably in a range of 1 to 20, and particularly preferably in a range of 1 to 12, and examples thereof include pyridylthio, 2-benzimidazolylthio, 2-benzoxazolylthio, and 2-benzothiazolylthio),

a sulfonyl group (the number of carbon atoms of the sulfonyl group is preferably in a range of 1 to 30, more preferably in a range of 1 to 20, and particularly preferably in a range of 1 to 12, and examples thereof include mesyl and tosyl), a sulfinyl group (the number of carbon atoms of the sulfinyl group is preferably in a range of 1 to 30, more preferably in a range of 1 to 20, and particularly preferably in a range of 1 to 12, and examples thereof include methanesulfinyl and benzenesulfinyl), an ureido group (the number of carbon atoms of the ureido group is preferably in a range of 1 to 30, more preferably in a range of 1 to 20, and particularly preferably in a range of 1 to 12, and examples thereof include ureido, methylureido, and phenylureido), a phosphoric acid amide group (the number of carbon atoms of the phosphoric acid amide group is preferably in a range of 1 to 30, more preferably in a range of 1 to 20, and particularly preferably in a range of 1 to 12, and examples thereof include diethyl phosphoric acid amide and phenyl phosphoric acid amide), a hydroxy group, a mercapto group, a halogen atom (such as a fluorine atom, a chlorine atom, a bromine atom, or an iodine atom, and a fluorine atom is more preferable),

a cyano group, a carboxy group, an oxo group, a nitro group, a hydroxamic acid group, a sulfino group, a hydrazine group, an imino group, a heterocyclic group (a 3- to 7-membered ring heterocyclic group is preferable, the hetero ring may be aromatic or non-aromatic, examples of a heteroatom constituting the hetero ring include a nitrogen atom, an oxygen atom, and a sulfur atom, the number of carbon atoms of the heterocyclic group is preferably in a range of 0 to 30 and more preferably in a range of 1 to 12, and specific examples thereof include imidazolyl, pyridyl, quinolyl, furyl, thienyl, piperidyl, morpholino, benzoxazolyl, benzimidazolyl, benzothiazolyl, carbazolyl, and azepinyl), a silyl group (the number of carbon atoms of the silyl group is preferably in a range of 3 to 40, more preferably in a range of 3 to 30, and particularly preferably in a range of 3 to 24, and examples thereof include trimethylsilyl and triphenylsilyl), and a silyloxy group (the number of carbon atoms of the silyloxy group is preferably in a range of 3 to 40, more preferably in a range of 3 to 30, and particularly preferably in a range of 3 to 24, and examples thereof include trimethylsilyloxy and triphenylsilyloxy). These substituents may further be substituted with any one or more substituents selected from the substituent group Z.

Further, in a case where a plurality of substituents are present at one structural site, these substituents may be linked to each other to form a ring or may be condensed with some or entirety of the structural site and form an aromatic ring or an unsaturated hetero ring.

In a case where a compound or a substituent includes an alkyl group or an alkenyl group, these may be linear or branched and may be substituted or unsubstituted. In addition, in a case where a compound or a substituent includes an aryl group or a heterocyclic group, these may be a single ring or a condensed ring and may be substituted or unsubstituted.

In the present specification, in a case where a group is described as only a substituent, the substituent group Z can be used as reference unless otherwise specified. Further, in a case where only the names of the respective groups are described (for example, a group is described as an “alkyl group”), the preferable range and the specific examples of the corresponding group in the substituent group Z are applied.

The molecular weight of the polyimide compound used in the present invention is preferably in a range of 10,000 to 1,000,000, more preferably in a range of 15,000 to 500,000, and still more preferably in a range of 20,000 to 200,000 as the weight-average molecular weight.

The molecular weight and the dispersity in the present specification are set to values measured using a gel permeation chromatography (GPC) method unless otherwise specified and the molecular weight is set to a weight-average molecular weight in terms of polystyrene. A gel including an aromatic compound as a repeating unit is preferable as a gel filling a column used for the GPC method and examples of the gel include a gel formed of a styrene-divinylbenzene copolymer. It is preferable that two to six columns are linked to each other and used. Examples of a solvent to be used include an ether-based solvent such as tetrahydrofuran and an amide-based solvent such as N-methylpyrrolidinone. It is preferable that measurement is performed at a flow rate of the solvent of 0.1 to 2 mL/min and most preferable that the measurement is performed at a flow rate thereof of 0.5 to 1.5 mL/min. In a case where the measurement is performed in the above-described range, a load is not applied to the apparatus and the measurement can be more efficiently performed. The measurement temperature is preferably in a range of 10° C. to 50° C. and most preferably in a range of 20° C. to 40° C. In addition, the column and the carrier to be used can be appropriately selected according to the physical properties of a polymer compound which is a target for measurement.

[Synthesis of Polyimide Compound]

The polyimide compound can be synthesized by performing condensation and polymerization of a bifunctional acid anhydride (tetracarboxylic dianhydride) having a specific structure and a specific diamine having a specific structure. Such methods can be performed by referring to the technique described in a general book (for example, “The Latest Polyimide ˜Fundamentals and Applications˜” edited by Toshio Imai and Rikio Yokota, NTS Inc., Aug. 25, 2010, pp. 3 to 49) as appropriate.

At least one tetracarboxylic dianhydride serving as a raw material in synthesis of the polyimide compound is represented by Formula (IV). It is preferable that all tetracarboxylic dianhydrides which are the raw materials are represented by Formula (IV).

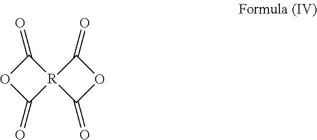

In Formula (IV), R has the same definition as that for R in Formula (I) and the preferred forms are the same as described above.

Specific examples of the tetracarboxylic dianhydride which can be used in the present invention include those shown below. In the description below, Ph represents phenyl.

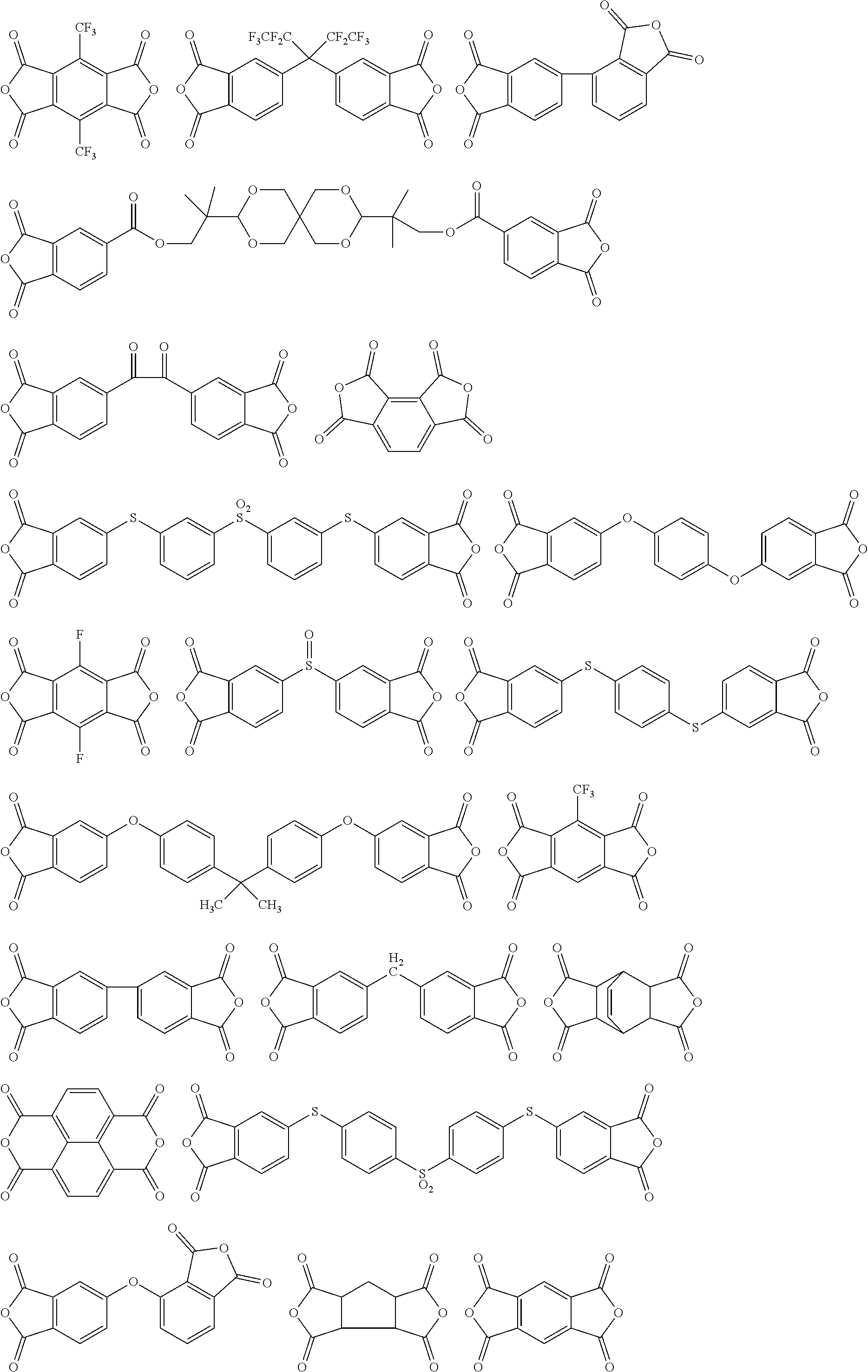

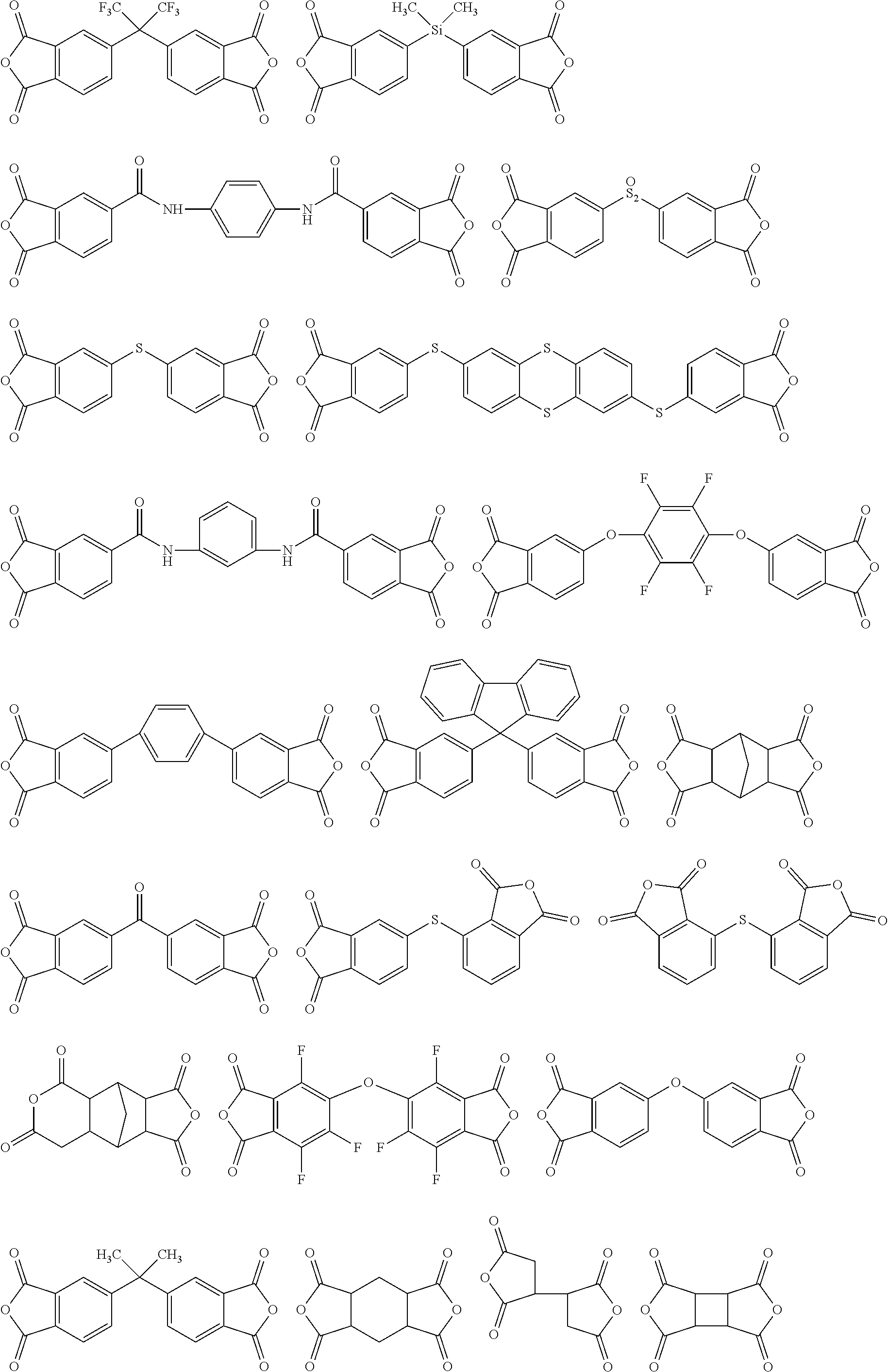

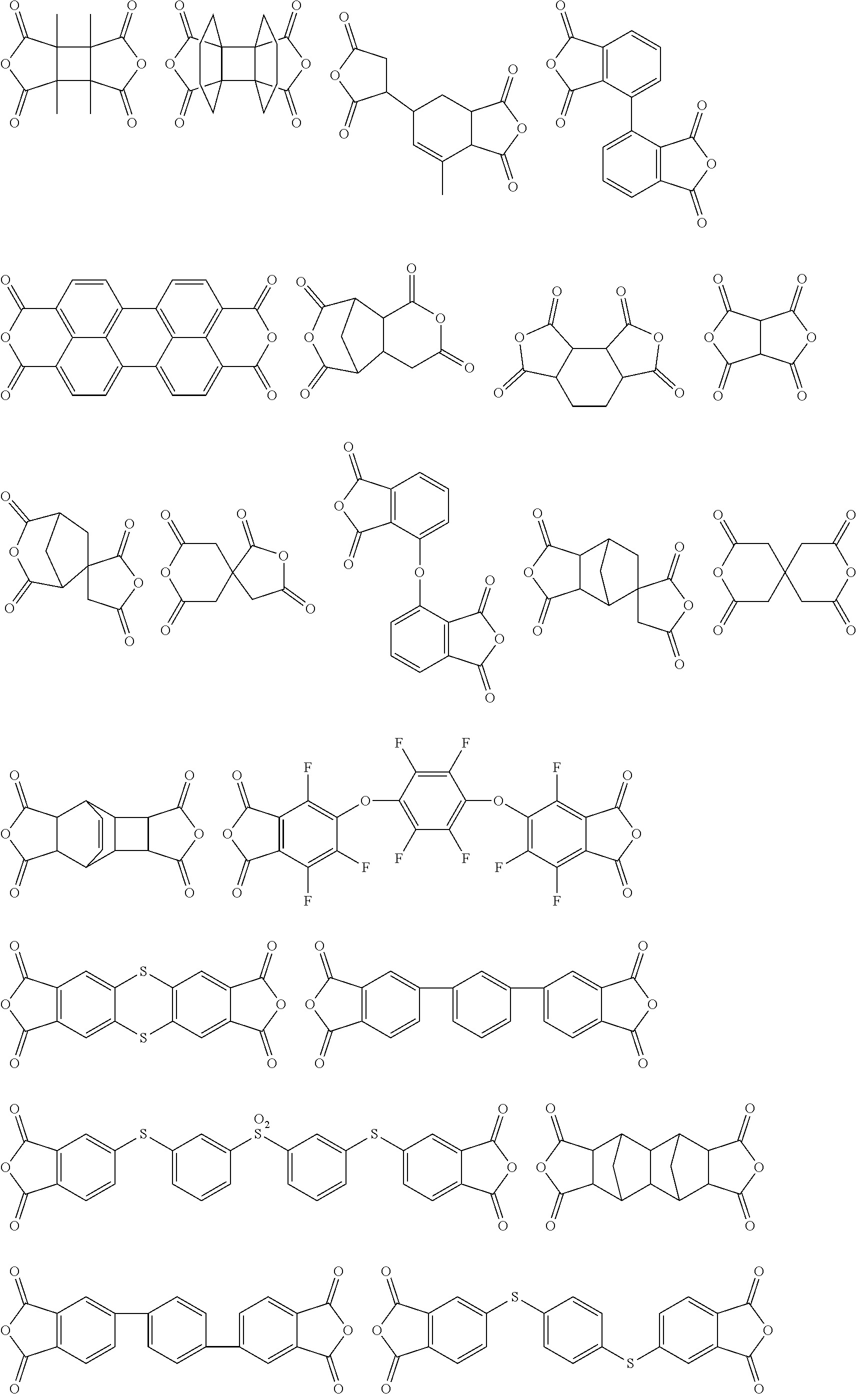

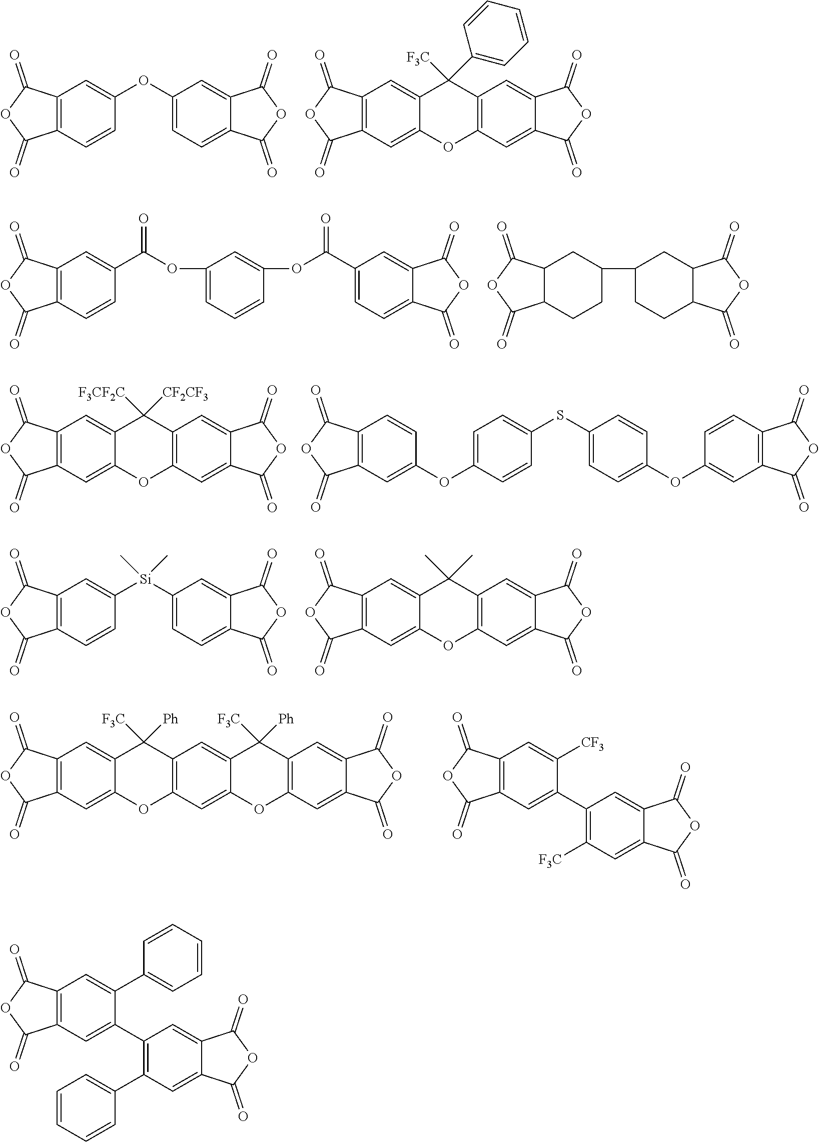

At least one diamine compound serving as the other raw material in synthesis of the polyimide compound used in the present invention is represented by Formula (V).

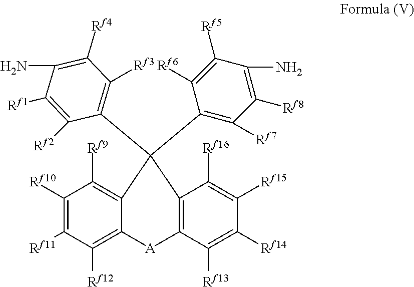

In Formula (V), A and R^{f1} to R^{f16} each have the same definition as that for A and R^{f1} to R^{f16} in Formula (I) and the preferred forms are the same as each other.

It is preferable that the diamine compound represented by Formula (V) is represented by Formula (V-a).

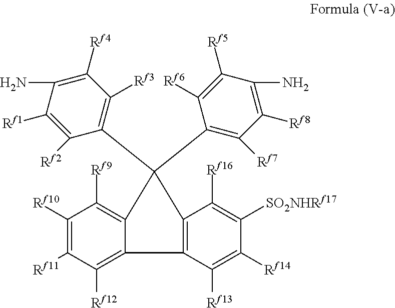

In Formula (V-a), R^{f1} to R^{f14}, R^{f16}, and R^{f17} each have the same definition as that for R^{f1} to R^{f14}, R^{f16}, and R^{f17} in Formula (I-a) and the preferred forms are the same as each other.

Specific preferred examples of the diamine compound represented by Formula (V) include those described below, but the present invention is not limited to these.

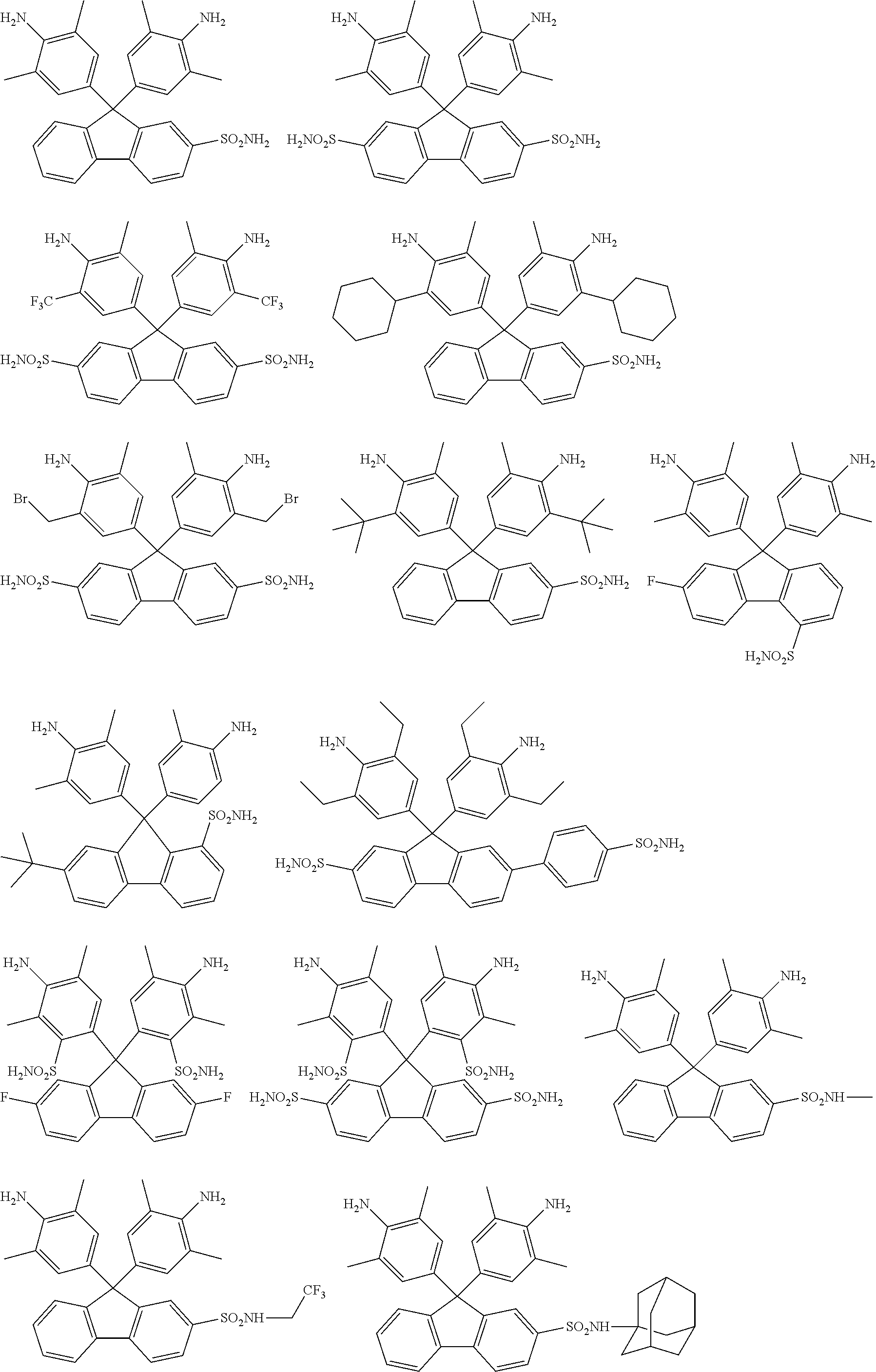

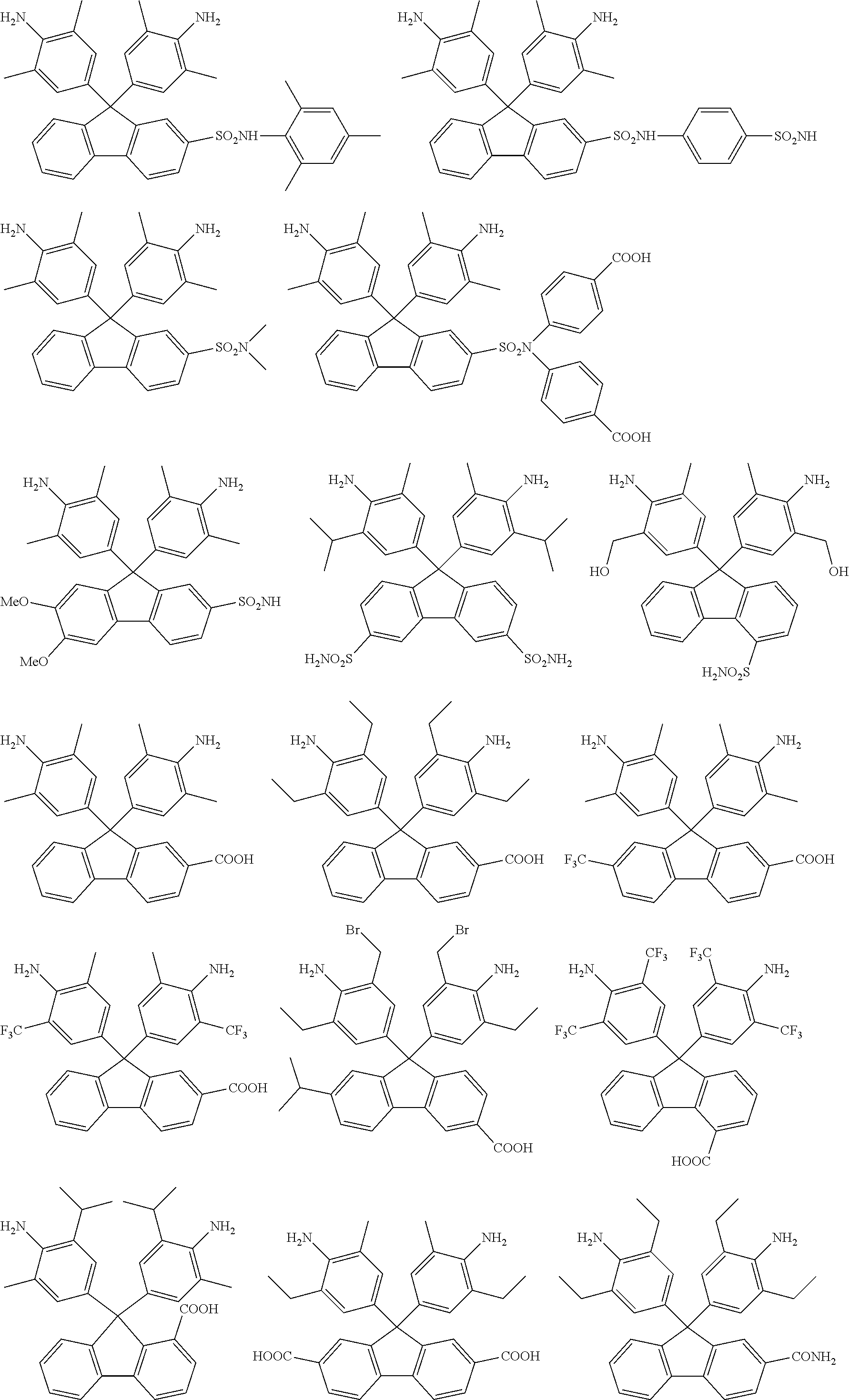

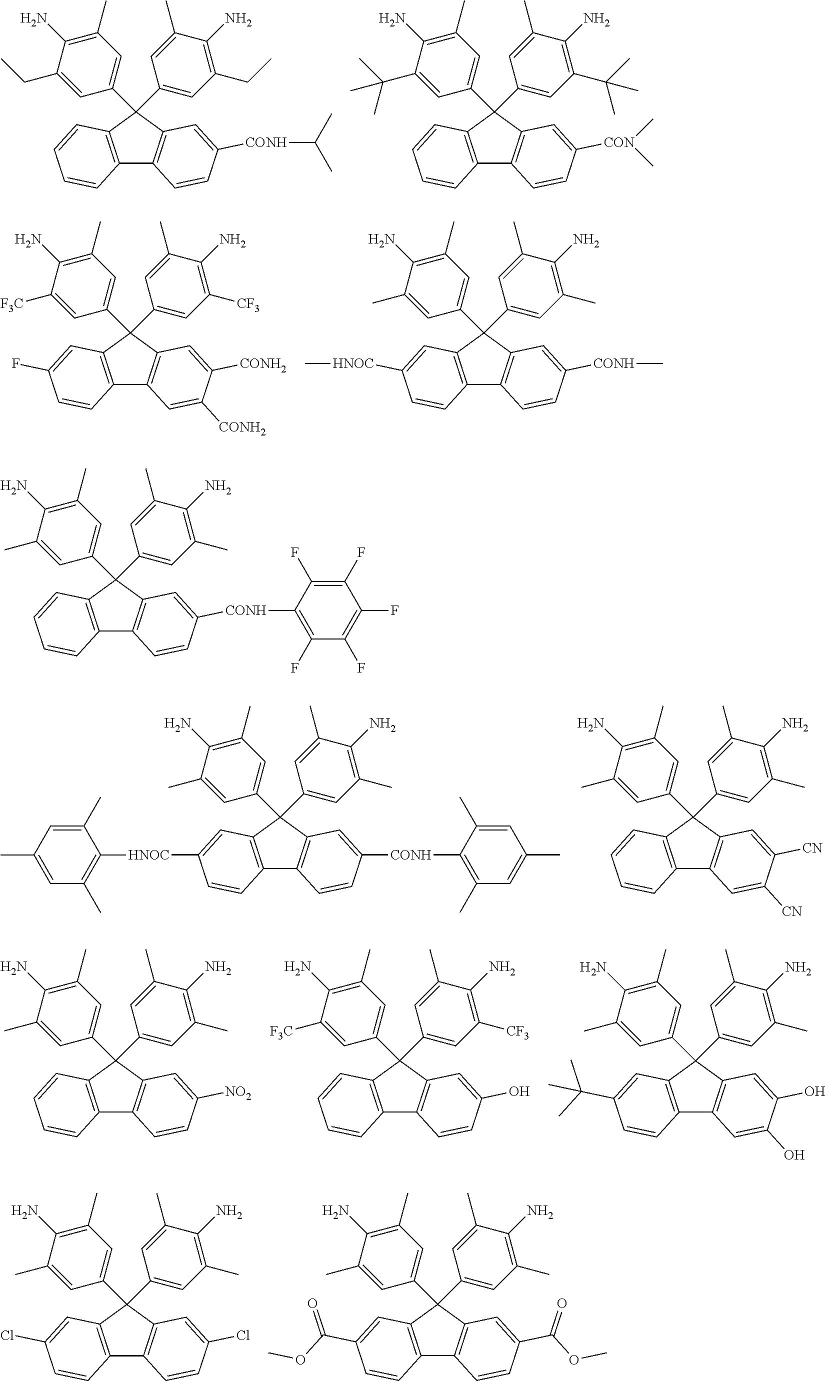

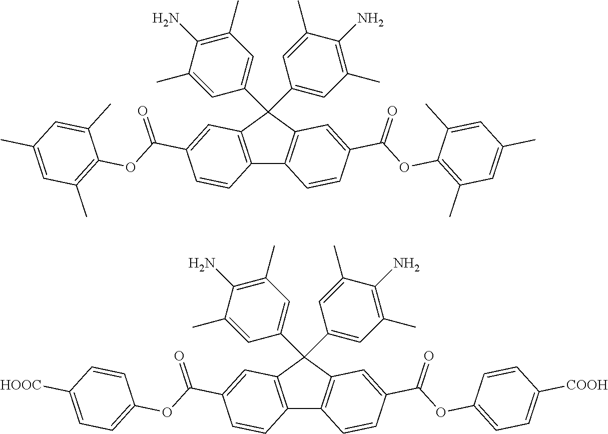

Further, in addition to the diamine compound represented by Formula (V), a diamine compound represented by Formula (VII-a) or (VII-b) may be used as the diamine compound serving as a raw material in the synthesis of the polyimide compound.

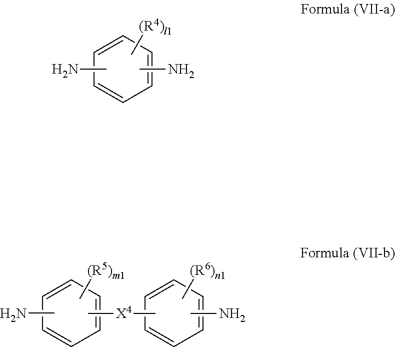

In Formula (VII-a), R⁴ and l1 each have the same definition as that for R⁴ and l1 in Formula (II-a) and the preferred forms are the same as each other.

In Formula (VII-b), R⁵, R⁶, X⁴, m1, and n1 each have the same definition as that for R⁵, R⁶, X⁴, m1, and n1 in Formula (II-b), and the preferable aspects thereof are the same as described above. Here, the diamine compound represented by Formula (VII-b) is not a diamine compound represented by Formula (V).

As the diamine compound represented by Formula (VII-a) or (VII-b), for example, diamine compounds shown below can be used.

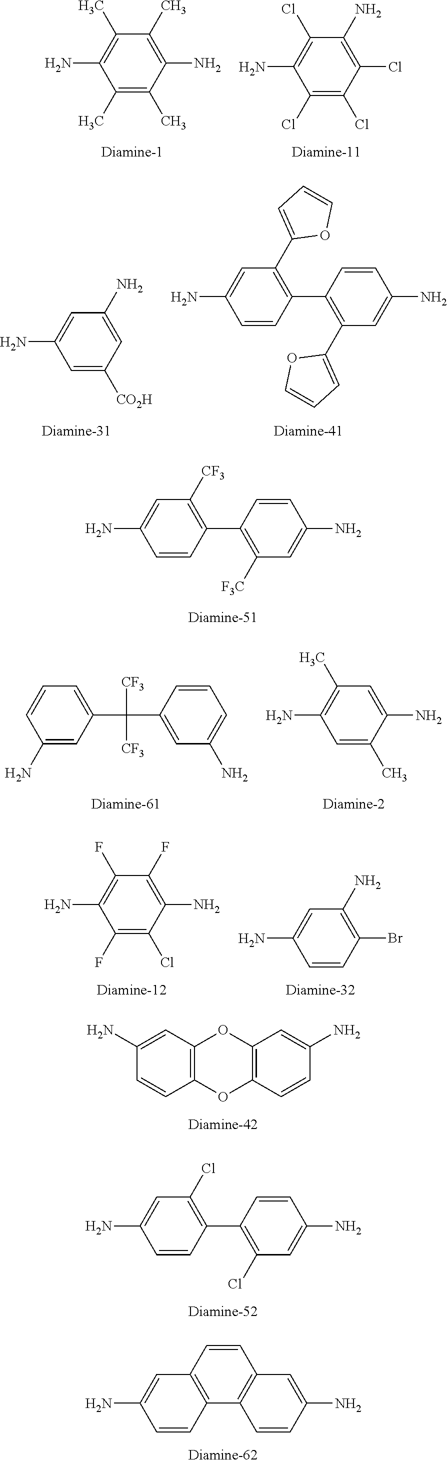

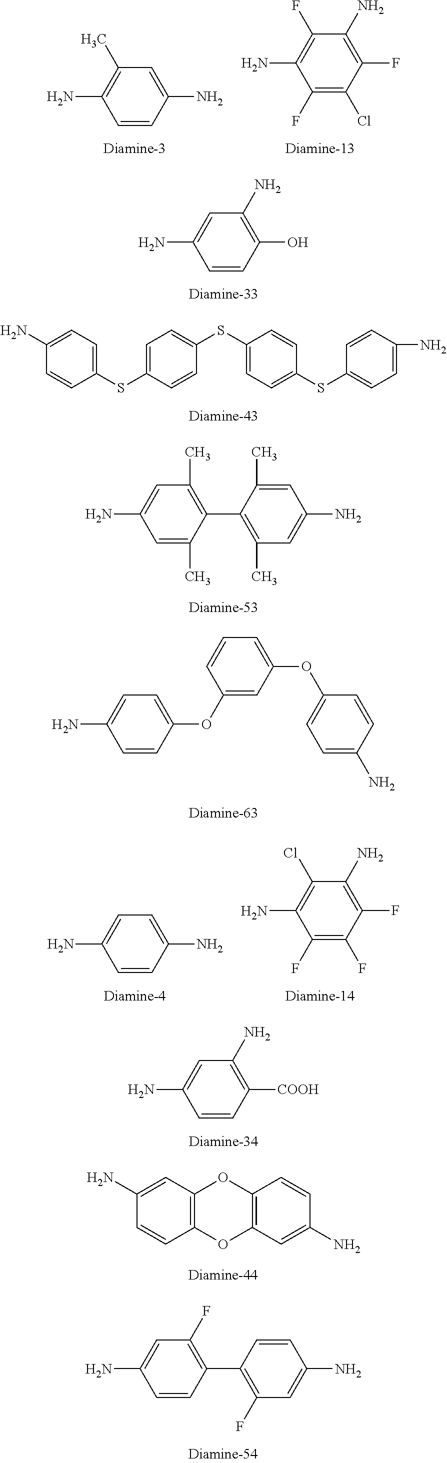

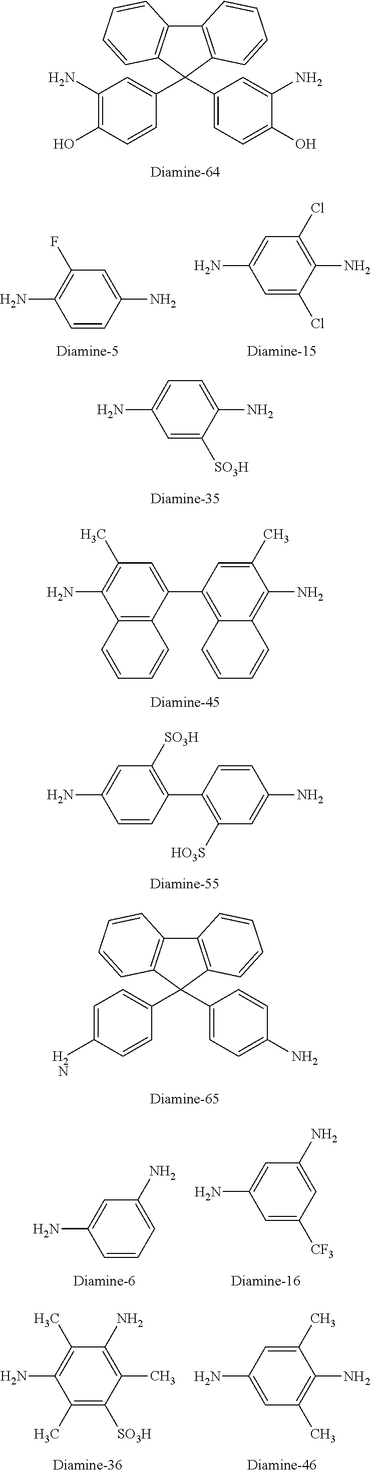

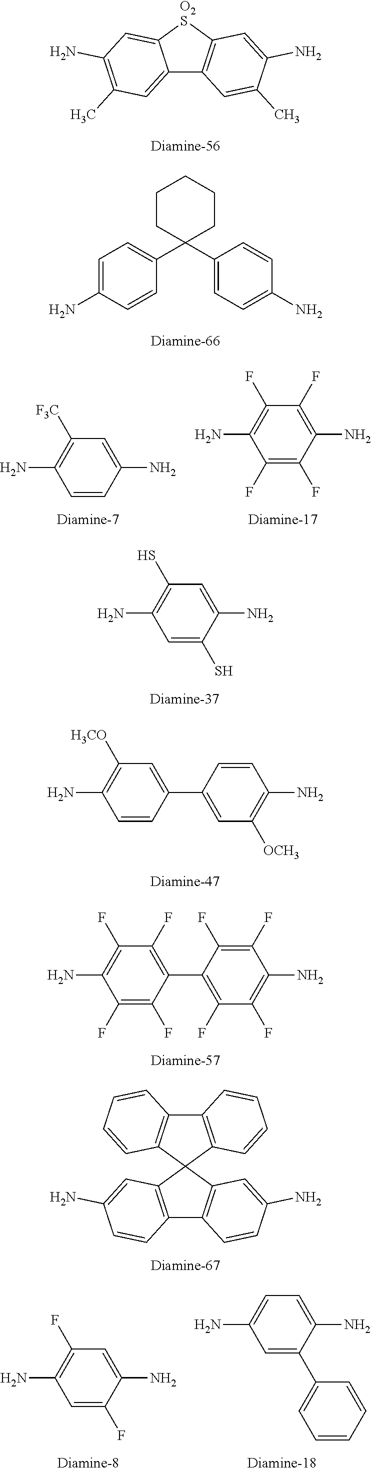

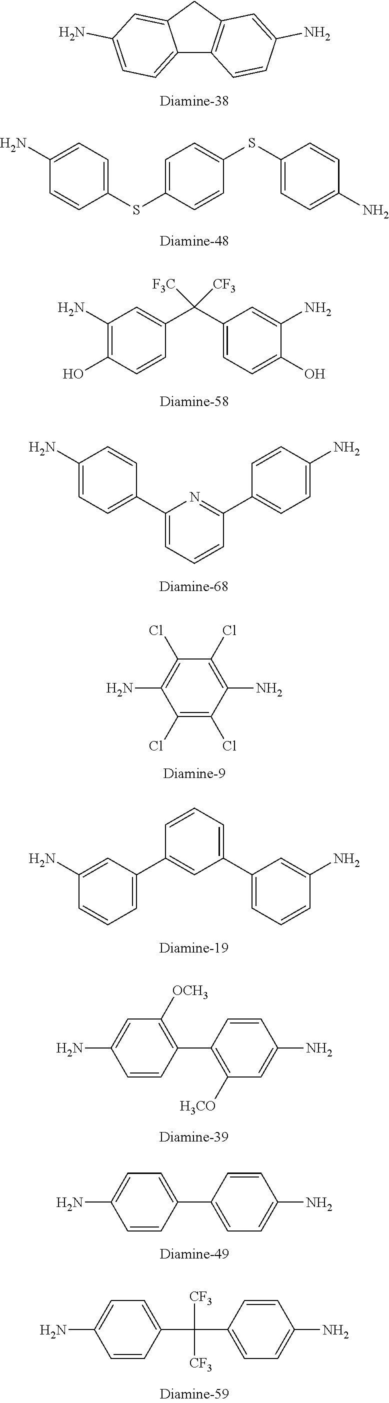

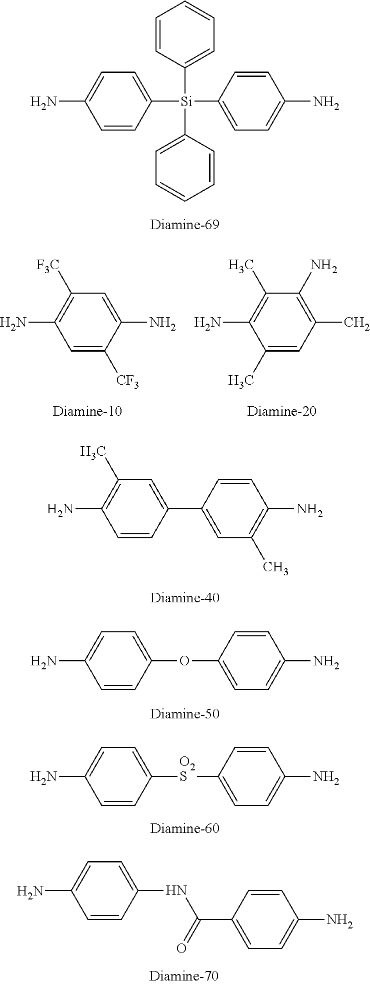

The monomer represented by Formula (IV) and the monomer represented by Formula (V), (VII-a), or (VII-b) may be used as oligomers or prepolymers in advance. The polyimide compound used in the present invention may be any of a block copolymer, a random copolymer, and a graft copolymer.

The polyimide compound used in the present invention can be obtained by mixing the above-described raw materials in a solvent and condensing and polymerizing the mixture using a typical method as described above.

The solvent is not particularly limited, and examples thereof include an ester such as methyl acetate, ethyl acetate, or butyl acetate; an aliphatic ketone such as acetone, methyl ethyl ketone, methyl isobutyl ketone, diacetone alcohol, cyclopentanone, or cyclohexanone; an ether such as diethylene glycol monomethyl ether, ethylene glycol dimethyl ether, dibutyl butyl ether, tetrahydrofuran, methyl cyclopentyl ether, or dioxane; an amide such as N-methylpyrrolidone, 2-pyrrolidone, dimethylformamide, dimethylimidazolidinone, or dimethylacetamide; and a sulfur-containing organic solvent such as dimethyl sulfoxide or sulfolane. These organic solvents can be suitably selected within the range in which a tetracarboxylic dianhydride serving as a reaction substrate, a diamine compound, polyamic acid which is a reaction intermediate, and a polyimide compound which is a final product can be dissolved. Among these, an ester (preferably butyl acetate), an aliphatic ketone (preferably methyl ethyl ketone, methyl isobutyl ketone, diacetone alcohol, cyclopentanone, or cyclohexanone), an ether (preferably diethylene glycol monomethyl ether or methyl cyclopentyl ether), an amide (preferably N-methylpyrrolidone), or a sulfur-containing organic solvent (preferably dimethyl sulfoxide or sulfolane) is preferable. In addition, these can be used alone or in combination of two or more kinds thereof.

The temperature of the polymerization reaction is not particularly limited and a temperature which can be typically employed for the synthesis of the polyimide compound can be employed. Specifically, the temperature is preferably in a range of −50° C. to 250° C., more preferably in a range of −25° C. to 225° C., still more preferably in a range of −0° C. to 200° C., and particularly preferably in a range of 20° C. to 190° C.

The polyimide compound can be obtained by imidizing the polyamic acid, which is generated by the above-described polymerization reaction, through a dehydration ring-closure reaction in a molecule. The method of the dehydration ring-closure can be performed by referring to the method described in a general book (for example, “The Latest Polyimide ˜Fundamentals and Applications˜” edited by Toshio Imai and Rikio Yokota, NTS Inc., Aug. 25, 2010, pp. 3 to 49). A thermal imidization method of performing heating in a temperature range of 120° C. to 200° C. and removing water generated as a by-product to the outside of the system for a reaction or a so-called chemical imidization method in which a dehydration condensation agent such as an acetic anhydride, dicyclohexylcarbodiimide, or triphenyl phosphite is used in the coexistence of a basic catalyst such as pyridine, triethylamine, or DBU (1,8-diazabicyclo[5.4.0]undec-7-ene) is suitably used.

In the present invention, the total concentration of the tetracarboxylic dianhydride and the diamine compound in the polymerization reaction solution of the polyimide compound is not particularly limited, but is preferably in a range of 5% to 70% by mass, more preferably in a range of 5% to 50% by mass, and still more preferably in a range of 5% to 30% by mass.

[Gas Separation Membrane]

[Gas Separation Composite Membrane]

The gas separation composite membrane which is a preferred form of the gas separation membrane of the present invention includes a gas separation layer formed by containing a specific polyimide compound on the upper side of the gas permeating support layer. It is preferable that the composite membrane is produced by coating at least a surface of a porous support with a coating solution (dope) containing the polyimide compound to form the gas separation layer. In the present specification, the concept “coating” includes the form of adhesion to a surface through immersion.

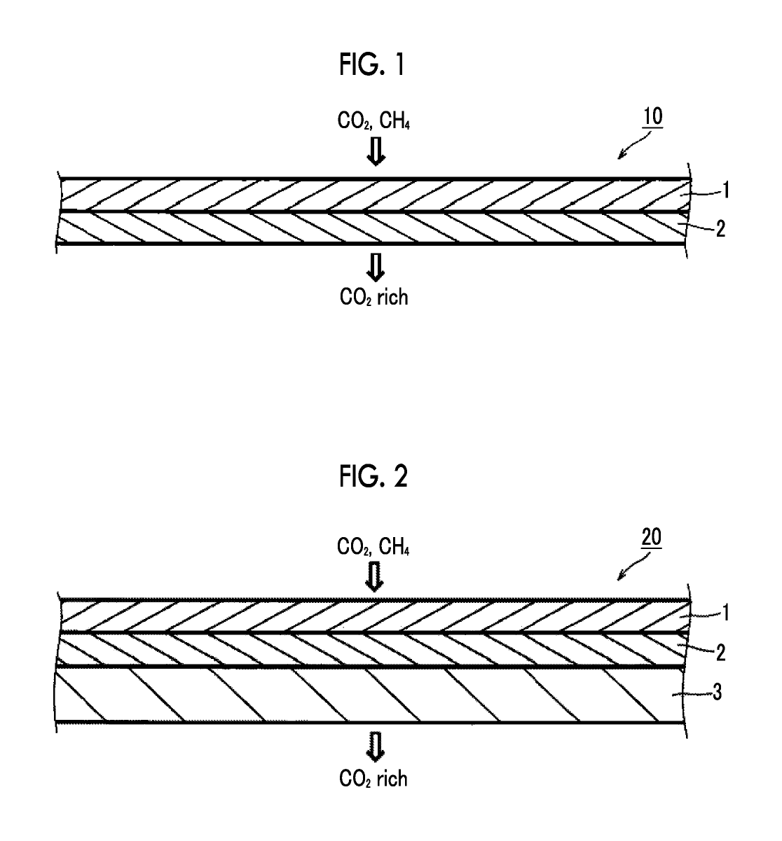

FIG. 1 is a longitudinal cross-sectional view schematically illustrating a gas separation composite membrane 10 which is a preferred embodiment of the present invention. The reference numeral 1 represents a gas separation layer and the reference numeral 2 represents a support layer formed of a porous layer. FIG. 2 is a cross-sectional view schematically illustrating a gas separation composite membrane 20 which is another preferred embodiment of the present invention. In the embodiment, a non-woven fabric layer 3 is added as a support layer in addition to the gas separation layer 1 and the porous layer 2. According to this embodiment, the gas permeating support layer includes the porous layer 2 on the gas separation layer 1 side and the non-woven fabric layer 3 on the opposite side thereof, and the gas separation layer 1 is provided on the upper side of the gas permeating support layer. In other words, the gas separation composite membrane 20 includes the gas separation layer 1, the porous layer 2, and the non-woven fabric layer 3 in this order.

FIGS. 1 and 2 illustrate the form of making permeating gas to be rich in carbon dioxide by selective permeation of carbon dioxide from a mixed gas of carbon dioxide and methane.

The expression “on the upper side of the support layer” in the present specification means that another layer may be interposed between the support layer and the gas separation layer. Further, in regard to the expressions related to up and down, the side where gas to be separated is supplied is set as “up” and the side where the separated gas is discharged is set as “down” unless otherwise specified.

The gas separation composite membrane of the present invention may be obtained by forming and disposing a gas separation layer on a surface or internal surface of the porous support (support layer) or can be obtained by simply forming a gas separation layer on at least a surface thereof to form a composite membrane. By forming a gas separation layer on at least a surface of the porous support, a composite membrane with an advantage of having excellent gas separation selectivity, excellent gas permeability, and mechanical strength can be obtained. As the membrane thickness of the gas separation layer, it is preferable that the gas separation layer is as thin as possible under conditions of imparting excellent gas permeability while maintaining the mechanical strength and the separation selectivity.

In the gas separation composite membrane, the thickness of the gas separation layer is not particularly limited. The thickness of the gas separation layer is preferably in a range of 0.01 to 5.0 μl and more preferably in a range of 0.05 to 2.0 μm.

The porous support (porous layer) which is preferably applied to the support layer is not particularly limited as long as the porous support is used for the purpose of imparting the mechanical strength and the excellent gas permeability, and the porous support may be formed of either of an organic material and an inorganic material. Among these, a porous membrane that contains an organic polymer is preferable. The thickness of the porous layer is in a range of 1 to 3,000 μm, preferably in a range of 5 to 500 μm, and more preferably in a range of 5 to 150 μm. The pore structure of this porous membrane has an average pore diameter of typically 10 μm or less, preferably 0.5 μm or less, and more preferably 0.2 μm or less. The porosity is preferably in a range of 20% to 90% and more preferably in a range of 30% to 80%.

Here, the support layer having the “gas permeability” means that the permeation rate of carbon dioxide is 1×10⁻⁵ cm³ (STP)/cm²·sec·cmHg (10 GPU) or greater in a case where carbon dioxide is supplied to the support layer (membrane formed of only the support layer) by setting the temperature to 40° C. and the total pressure on the side to which gas is supplied to 4 MPa. Further, in regard to the gas permeability of the support layer, the permeation rate of carbon dioxide is preferably 3×10⁻⁵ cm³ (STP)/cm²·sec·cmHg (30 GPU) or greater, more preferably 100 GPU or greater, and still more preferably 200 GPU or greater in a case where carbon dioxide is supplied by setting the temperature to 40° C. and the total pressure on the side to which gas is supplied to 4 MPa. Examples of the material of the porous membrane include conventionally known polymers, for example, a polyolefin-based resin such as polyethylene or polypropylene; a fluorine-containing resin such as polytetrafluoroethylene, polyvinyl fluoride, or polyvinylidene fluoride; and various resins such as polystyrene, cellulose acetate, polyurethane, polyacrylonitrile, polyphenylene oxide, polysulfone, polyether sulfone, polyimide, and polyaramid. As the shape of the porous membrane, any shape from among a flat plate shape, a spiral shape, a tabular shape, and a hollow fiber shape can be employed.

In the gas separation composite membrane, it is preferable that a support is formed in the lower portion of the support layer that forms the gas separation membrane for imparting mechanical strength. Examples of such a support include woven fabric, non-woven fabric, and a net. Among these, from the viewpoints of membrane forming properties and the cost, non-woven fabric is suitably used. As the non-woven fabric, fibers formed of polyester, polypropylene, polyacrylonitrile, polyethylene, and polyamide may be used alone or in combination of plural kinds thereof. The non-woven fabric can be produced by papermaking main fibers and binder fibers which are uniformly dispersed in water using a circular net or a long net and then drying the fibers with a dryer. Moreover, for the purpose of removing a nap or improving mechanical properties, it is preferable that thermal pressing processing is performed on the non-woven fabric by interposing the non-woven fabric between two rolls.

<Method of Producing Gas Separation Composite Membrane>

As a method of producing the composite membrane of the present invention, a production method which includes coating a support with a coating solution containing the above-described polyimide compound to form a gas separation layer is preferable. The content of the polyimide compound in the coating solution is not particularly limited, but is preferably in a range of 0.1% to 30% by mass and more preferably in a range of 0.5% to 10% by mass. In a case where the content of the polyimide compound is extremely small, defects are highly likely to occur in the surface layer contributing to gas separation because the coating solution easily permeates to the underlayer at the time of formation of the gas separation layer on the porous support. In addition, in a case where the content of the polyimide compound is extremely large, there is a possibility that the gas permeability is degraded because holes are filled with the coating solution at a high concentration at the time of formation of the gas separation layer on the porous support. The gas separation membrane of the present invention can be appropriately produced by adjusting the molecular weight of the polymer, the structure, and the composition of the gas separation layer and the viscosity of the solution.

The organic solvent serving as a medium of the coating solution is not particularly limited, and examples thereof include hydrocarbon such as n-hexane or n-heptane; an ester such as methyl acetate, ethyl acetate, or butyl acetate; alcohol such as methanol, ethanol, n-propanol, isopropanol, n-butanol, isobutanol, or tert-butanol; an aliphatic ketone such as acetone, methyl ethyl ketone, methyl isobutyl ketone, diacetone alcohol, cyclopentanone, or cyclohexanone; an ether such as ethylene glycol, diethylene glycol, triethylene glycol, glycerin, propylene glycol, ethylene glycol monomethyl ether, ethylene glycol monoethyl ether, propylene glycol methyl ether, dipropylene glycol methyl ether, tripropylene glycol methyl ether, ethylene glycol phenyl ether, propylene glycol phenyl ether, diethylene glycol monomethyl ether, diethylene glycol monoethyl ether, diethylene glycol monobutyl ether, triethylene glycol monomethyl ether, triethylene glycol monoethyl ether, dibutyl butyl ether, tetrahydrofuran, methyl cyclopentyl ether, or dioxane; and N-methylpyrrolidone, 2-pyrrolidone, dimethylformamide, dimethylimidazolidinone, dimethyl sulfoxide, and dimethyl acetamide. These organic solvents are appropriately selected within the range that does not adversely affect the support through erosion or the like, and an ester (preferably butyl acetate), an alcohol (preferably methanol, ethanol, isopropanol, or isobutanol), an aliphatic ketone (preferably methyl ethyl ketone, methyl isobutyl ketone, diacetone alcohol, cyclopentanone, or cyclohexanone), and an ether (preferably ethylene glycol, diethylene glycol monomethyl ether, or methyl cyclopentyl ether) are preferable and an aliphatic ketone, an alcohol, and an ether are more preferable. Further, these may be used alone or in combination of two or more kinds thereof.

(Another Layer Between Support Layer and Gas Separation Layer)

In the gas separation composite membrane, another layer may be present between the support layer and the gas separation layer. Preferred examples of another layer include a siloxane compound layer. By providing a siloxane compound layer, unevenness of the outermost surface of the support layer can be made to be smooth and the thickness of the gas separation layer is easily reduced. Examples of a siloxane compound that forms the siloxane compound layer include a compound in which the main chain is formed of polysiloxane and a compound having a siloxane structure and a non-siloxane structure in the main chain.

The “siloxane compound” in the present specification indicates an organopolysiloxane compound unless otherwise noted.

—Siloxane Compound Whose Main Chain is Formed of Polysiloxane—

As the siloxane compound which can be used for the siloxane compound layer and whose main chain is formed of polysiloxane, one or two or more kinds of polyorganopolysiloxanes represented by Formula (1) or (2) may be exemplified. Further, these polyorganopolysiloxanes may form a crosslinking reactant. As the crosslinking reactant, a compound in the form of the compound represented by Formula (1) being crosslinked by a polysiloxane compound having groups linked to each other by reacting with a reactive group X^{S} of Formula (1) at both terminals is exemplified.

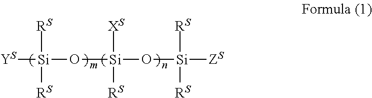

In Formula (1), R^{S} represents a non-reactive group. Specifically, it is preferable that R^{S} represents an alkyl group (an alkyl group having preferably 1 to 18 carbon atoms and more preferably 1 to 12 carbon atoms) or an aryl group (an aryl group having preferably 6 to 15 carbon atoms and more preferably 6 to 12 carbon atoms; and more preferably phenyl).

X^{S} represents a reactive group, and it is preferable that X^{S} represents a group selected from a hydrogen atom, a halogen atom, a vinyl group, a hydroxyl group, and a substituted alkyl group (an alkyl group having preferably 1 to 18 carbon atoms and more preferably 1 to 12 carbon atoms).

Y^{S} and Z^{S} each have the same definition as that for R^{S} or X^{S} described above.

m represents an integer of 1 or greater and preferably 1 to 100,000.

n represents an integer of 0 or greater and preferably 0 to 100,000.

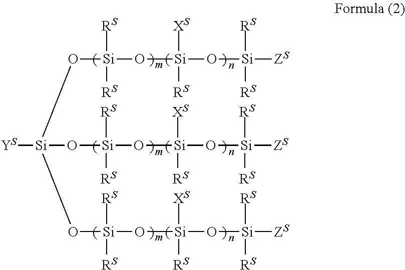

In Formula (2), X^{S}, Y^{S}, Z^{S}, R^{S}, m, and n each have the same definition as that for X^{S}, Y^{S}, Z^{S}, R^{S}, m, and n in Formula (1).

In Formulae (1) and (2), in a case where the non-reactive group R^{S} represents an alkyl group, examples of the alkyl group include methyl, ethyl, hexyl, octyl, decyl, and octadecyl. Further, in a case where the non-reactive group R represents a fluoroalkyl group, examples of the fluoroalkyl group include —CH₂CH₂CF₃, and —CH₂CH₂C₆F₁₃.

In Formulae (1) and (2), in a case where the reactive group X^{S} represents a substituted alkyl group, examples of the alkyl group include a hydroxyalkyl group having 1 to 18 carbon atoms, an aminoalkyl group having 1 to 18 carbon atoms, a carboxyalkyl group having 1 to 18 carbon atoms, a cycloalkyl group having 1 to 18 carbon atoms, a glycidoxyalkyl group having 1 to 18 carbon atoms, a glycidyl group, an epoxycyclohexylalkyl group having 7 to 16 carbon atoms, a (1-oxacyclobutane-3-yl)alkyl group having 4 to 18 carbon atoms, a methacryloxyalkyl group, and a mercaptoalkyl group.

The number of carbon atoms of the alkyl group constituting the hydroxyalkyl group is preferably an integer of 1 to 10, and examples of the hydroxyalkyl group include —CH₂CH₂CH₂OH.

The number of carbon atoms of the alkyl group constituting the aminoalkyl group is preferably an integer of 1 to 10, and examples of the aminoalkyl group include —CH₂CH₂CH₂NH₂.

The number of carbon atoms of the alkyl group constituting the carboxyalkyl group is preferably an integer of 1 to 10, and examples of the carboxyalkyl group include —CH₂CH₂CH₂COOH.

The number of carbon atoms of the alkyl group constituting the chloroalkyl group is preferably an integer of 1 to 10, and preferred examples of the chloroalkyl group include —CH₂Cl.

The number of carbon atoms of the alkyl group constituting the glycidoxyalkyl group is preferably an integer of 1 to 10, and preferred examples of the glycidoxyalkyl group include 3-glycidyloxypropyl.

The number of carbon atoms of the epoxycyclohexylalkyl group having 7 to 16 carbon atoms is preferably an integer of 8 to 12.

The number of carbon atoms of the (1-oxacyclobutane-3-yl)alkyl group having 4 to 18 carbon atoms is preferably an integer of 4 to 10.

The number of carbon atoms of the alkyl group constituting the methacryloxyalkyl group is preferably an integer of 1 to 10, and examples of the methacryloxyalkyl group include —CH₂CH₂CH₂—OOC—C(CH₃)═CH₂.

The number of carbon atoms of the alkyl group constituting the mercaptoalkyl group is preferably an integer of 1 to 10, and examples of the mercaptoalkyl group include —CH₂CH₂CH₂SH.

It is preferable that m and n represent a number in which the molecular weight of the compound is in a range of 5,000 to 1,000,000.

In Formulae (1) and (2), distribution of a reactive group-containing siloxane unit (in the formulae, a constitutional unit whose number is represented by n) and a siloxane unit (in the formulae, a constitutional unit whose number is represented by m) which does not have a reactive group is not particularly limited. That is, in Formulae (1) and (2), the (Si(R^{S})(R^{S})—O) unit and the (Si(R^{S})(X^{S})—O) unit may be randomly distributed.

—Compound Having Siloxane Structure and Non-Siloxane Structure in Main Chain—

Examples of the compound which can be used for the siloxane compound layer and has a siloxane structure and a non-siloxane structure in the main chain include compounds represented by Formulae (3) to (7).

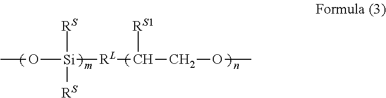

In Formula (3), R^{S}, m, and n each have the same definition as that for R^{S}, m, and n in Formula (1). R^{L} represents —O— or —CH₂— and R^{S1} represents a hydrogen atom or methyl. It is preferable that both terminals of Formula (3) are each independently formed of an amino group, a hydroxyl group, a carboxy group, a trimethylsilyl group, an epoxy group, a vinyl group, a hydrogen atom, or a substituted alkyl group.

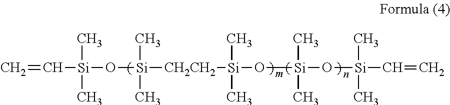

In Formula (4), m and n each have the same definition as that for m and n in Formula (1).

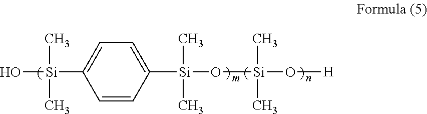

In Formula (5), m and n each have the same definition as that for m and n in Formula (1).

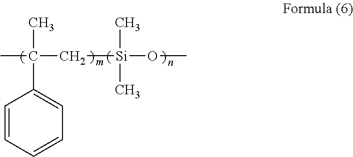

In Formula (6), m and n each have the same definition as that for m and n in Formula (1). It is preferable that both terminals of Formula (6) are each independently bonded to an amino group, a hydroxyl group, a carboxy group, a trimethylsilyl group, an epoxy group, a vinyl group, a hydrogen atom, or a substituted alkyl group.

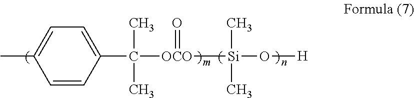

In Formula (7), m and n each have the same definition as that for m and n in Formula (1). It is preferable that both terminals of Formula (7) are each independently bonded to an amino group, a hydroxyl group, a carboxy group, a trimethylsilyl group, epoxy, a vinyl group, a hydrogen atom, or a substituted alkyl group.

In Formulae (3) to (7), distribution of a siloxane structural unit and a non-siloxane structural unit may be randomly distributed.

It is preferable that the compound having a siloxane structure and a non-siloxane structure in the main chain contains 50% by mole or greater of the siloxane structural unit and more preferable that the compound contains 70% by mole or greater of the siloxane structural unit with respect to the total molar amount of all repeating structural units.

From the viewpoint of achieving the balance between durability and reduction in membrane thickness, the weight-average molecular weight of the siloxane compound used for the siloxane compound layer is preferably in a range of 5,000 to 1,000,000. The method of measuring the weight-average molecular weight is as described above.

Further, preferred examples of the siloxane compound constituting the siloxane compound layer are as follows.

Preferred examples thereof include one or two or more selected from polydimethylsiloxane, polymethylphenylsiloxane, polydiphenylsiloxane, a polysulfone/polyhydroxystyrene/polydimethylsiloxane copolymer, a dimethylsiloxane/methylvinylsiloxane copolymer, a dimethylsiloxane/diphenylsiloxane/methylvinylsiloxane copolymer, a methyl-3,3,3-trifluoropropylsiloxane/methylvinylsiloxane copolymer, a dimethylsiloxane/methylphenylsiloxane/methylvinylsiloxane copolymer, a vinyl terminated diphenylsiloxane/dimethylsiloxane copolymer, vinyl terminated polydimethylsiloxane, H terminated polydimethylsiloxane, and a dimethylsiloxane/methylhydroxysiloxane copolymer. Further, these compounds also include the forms of forming crosslinking reactants.

In the gas separation composite membrane, from the viewpoints of smoothness and gas permeability, the thickness of the siloxane compound layer is preferably in a range of 0.01 to 5 μm and more preferably in a range of 0.05 to 1 μm.

Further, the gas permeability of the siloxane compound layer at 40° C. and 4 MPa is preferably 100 GPU or greater, more preferably 300 GPU or greater, and still more preferably 1,000 GPU or greater in terms of the permeation rate of carbon dioxide.

[Gas Separation Asymmetric Membrane]

The gas separation membrane may be an asymmetric membrane. The asymmetric membrane can be formed according to a phase inversion method using a solution containing a polyimide compound. The phase inversion method is a known method of allowing a polymer solution to be brought into contact with a coagulating liquid for phase inversion to form a membrane, and a so-called dry-wet method is suitably used in the present invention. The dry-wet method is a method of forming a porous layer by evaporating a solution on the surface of a polymer solution which is made to have a membrane shape to form a thin compact layer, immersing the compact layer in a coagulating liquid (the coagulating liquid indicates a solvent which is compatible with a solvent of a polymer solution and in which a polymer is insoluble), and forming fine pores using a phase separation phenomenon that occurs at this time, and this method is suggested by Loeb and Sourirajan (for example, the specification of U.S. Pat. No. 3,133,132A).

In the gas separation asymmetric membrane of the present invention, the thickness of the surface layer contributing to gas separation, which is referred to as a compact layer or a skin layer, is not particularly limited. The thickness of the surface layer is preferably in a range of 0.01 to 5.0 μm and more preferably in a range of 0.05 to 1.0 μm from the viewpoint of imparting practical gas permeability. In addition, the porous layer positioned in the lower portion of the compact layer plays a role of decreasing gas permeability resistance and imparting the mechanical strength at the same time, and the thickness thereof is not particularly limited as long as self-supporting properties as an asymmetric membrane are imparted. The thickness thereof is preferably in a range of 5 to 500 μm, more preferably in a range of 5 to 200 μm, and still more preferably in a range of 5 to 100 μm.

The gas separation asymmetric membrane of the present invention may be a flat membrane or a hollow fiber membrane. An asymmetric hollow fiber membrane can be produced by a dry-wet spinning method. The dry-wet spinning method is a method of producing an asymmetric hollow fiber membrane by applying a dry-wet method to a polymer solution which is discharged from a spinning nozzle in a target shape which is a hollow fiber shape. More specifically, the dry-wet spinning method is a method in which a polymer solution is discharged from a nozzle in a target shape which is a hollow fiber shape and passes through air or a nitrogen gas atmosphere immediately after the discharge. Thereafter, an asymmetric structure is formed through immersion in a coagulating liquid which does not substantially dissolve a polymer and is compatible with a solvent of the polymer solution. Further, the membrane is dried and subjected to a heat treatment as necessary, thereby producing a separation membrane.

It is preferable that the solution viscosity of the solution containing a polyimide compound which is discharged from a nozzle is in a range of 2 to 17,000 Pa·s, preferably 10 to 1,500 Pa·s, and particularly preferably in a range of 20 to 1,000 Pa·s at the discharge temperature (for example, 10° C.) from a viewpoint of stably obtaining the shape after the discharge such as a hollow fiber shape or the like. It is preferable that immersion of a membrane in a coagulating liquid is carried out by immersing the membrane in a primary coagulating liquid to be solidified to the extent that the shape of a membrane such as a hollow fiber shape can be maintained, winding the membrane around a guide roll, immersing the membrane in a secondary coagulating liquid, and sufficiently solidifying the whole membrane. It is efficient that the solidified membrane is dried after the coagulating liquid is substituted with a solvent such as hydrocarbon. It is preferable that the heat treatment for drying the membrane is performed at a temperature lower than the softening point or the secondary transition point of the used polyimide compound.

In the gas separation membrane, a siloxane compound layer may be provided as a protective layer by being brought into contact with the gas separation layer.

[Use and Properties of Gas Separation Membrane]

The gas separation membrane (the composite membrane and the asymmetric membrane) can be suitably used according to a gas separation recovery method and a gas separation purification method. For example, a gas separation membrane which is capable of efficiently separating specific gas from a gas mixture containing gas, for example, hydrocarbon such as hydrogen, helium, carbon monoxide, carbon dioxide, hydrogen sulfide, oxygen, nitrogen, ammonia, a sulfur oxide, a nitrogen oxide, methane, or ethane; unsaturated hydrocarbon such as propylene; or a perfluoro compound such as tetrafluoroethane can be obtained. Particularly, it is preferable that a gas separation membrane selectively separating carbon dioxide from a gas mixture containing carbon dioxide and hydrocarbon (methane) is obtained.

In addition, in a case where gas subjected to a separation treatment is a mixed gas of carbon dioxide and methane, the permeation rate of the carbon dioxide in the mixed gas at 40° C. and 5 MPa is preferably greater than 20 GPU, more preferably greater than 30 GPU, still more preferably in a range of 35 GPU to 500 GPU, and even still more preferably greater than 60 and 300 GPU or less. The ratio (R_{CO2}/R_{CH4}) between permeation rates of carbon dioxide and methane is preferably 15 or greater, and more preferably 20 or greater. R_{CO2} represents the permeation rate of carbon dioxide and R_{CH4} represents the permeation rate of methane.

Further, 1 GPU is 1×10⁻⁶ cm³ (STP)/cm²·cm·sec·cmHg.

[Other Components and the Like]

Various polymer compounds can also be added to the gas separation layer of the gas separation membrane in order to adjust the physical properties of the membrane. As the polymer compounds, an acrylic polymer, a polyurethane resin, a polyamide resin, a polyester resin, an epoxy resin, a phenol resin, a polycarbonate resin, a polyvinyl butyral resin, a polyvinyl formal resin, shellac, a vinyl-based resin, an acrylic resin, a rubber-based resin, waxes, and other natural resins can be used. Further, these may be used in combination of two or more kinds thereof.

Further, a non-ionic surfactant, a cationic surfactant, or an organic fluoro compound can be added to the gas separation membrane of the present invention in order to adjust the physical properties of the liquid.

Specific examples of the surfactant include anionic surfactants such as alkyl benzene sulfonate, alkyl naphthalene sulfonate, higher fatty acid salts, sulfonate of higher fatty acid ester, sulfuric ester salts of higher alcohol ether, sulfonate of higher alcohol ether, alkyl carboxylate of higher alkyl sulfonamide, and alkyl phosphate; and non-ionic surfactants such as polyoxyethylene alkyl ether, polyoxyethylene alkyl phenyl ether, polyoxyethylene fatty acid ester, sorbitan fatty acid ester, an ethylene oxide adduct of acetylene glycol, an ethylene oxide adduct of glycerin, and polyoxyethylene sorbitan fatty acid ester. Other examples thereof include amphoteric surfactants such as alkyl betaine and amide betaine; a silicon-based surfactant; and a fluorine-based surfactant. The surfactant can be suitably selected from known surfactants and derivatives thereof in the related art.

Further, the gas separation layer of the gas separation membrane may contain a polymer dispersant. Specific examples of the polymer dispersant include polyvinyl pyrrolidone, polyvinyl alcohol, polyvinyl methyl ether, polyethylene oxide, polyethylene glycol, polypropylene glycol, and polyacrylamide. Among these, polyvinyl pyrrolidone is preferably used.

The conditions of forming the gas separation membrane of the present invention are not particularly limited. The temperature thereof is preferably in a range of −30° C. to 100° C., more preferably in a range of −10° C. to 80° C., and particularly preferably in a range of 5° C. to 50° C.

Gas such as air or oxygen may be allowed to coexist during membrane formation, and it is desired that the membrane is formed under an inert gas atmosphere.

In the gas separation membrane, the content of the polyimide compound in the gas separation layer is not particularly limited as long as desired gas separation performance can be obtained. From the viewpoint of further improving gas separation performance, the content of the polyimide compound in the gas separation layer is preferably 20% by mass or greater, more preferably 40% by mass or greater, still more preferably 60% by mass or greater, and particularly preferably 70% by mass or greater. Further, the content of the polyimide compound in the gas separation layer may be 100% by mass and is typically 99% by mass or less.

[Method of Separating Gas Mixture]

The gas separation method of the present invention is a method of separating specific gas from a mixed gas containing two or more components using the gas separation membrane of the present invention. The gas separation method includes selectively permeating carbon dioxide from the mixed gas containing carbon dioxide and methane. The gas pressure at the time of gas separation is preferably in a range of 0.5 MPa to 10 MPa, more preferably in a range of 1 MPa to 10 MPa, and still more preferably in a range of 2 MPa to 7 MPa. Further, the temperature for separating gas is preferably in a range of −30° C. to 90° C. and more preferably in a range of 15° C. to 70° C. In the mixed gas containing carbon dioxide and methane gas, the mixing ratio of carbon dioxide to methane gas is not particularly limited. The mixing ratio thereof (carbon dioxide:methane gas) is preferably in a range of 1:99 to 99:1 (volume ratio) and more preferably in a range of 5:95 to 90:10.

[Gas Separation Module and Gas Separator]

A gas separation module can be prepared using the gas separation membrane of the present invention. Examples of the module include a spiral type module, a hollow fiber type module, a pleated module, a tubular module, and a plate and frame type module.

Moreover, it is possible to obtain a gas separator having means for performing separation and recovery of gas or performing separation and purification of gas by using the gas separation composite membrane of the present invention or the gas separation module. The gas separation composite membrane of the present invention may be applied to a gas separation and recovery device which is used together with an absorption liquid described in JP2007-297605A according to a membrane/absorption hybrid method.

### EXAMPLES

Hereinafter, the present invention will be described in detail with reference to examples, but the present invention is not limited these examples.

### Synthesis Example

<Synthesis of Polyimide PI-01>

Polyimide PI-01 formed of the following repeating unit was synthesized according to the following scheme.

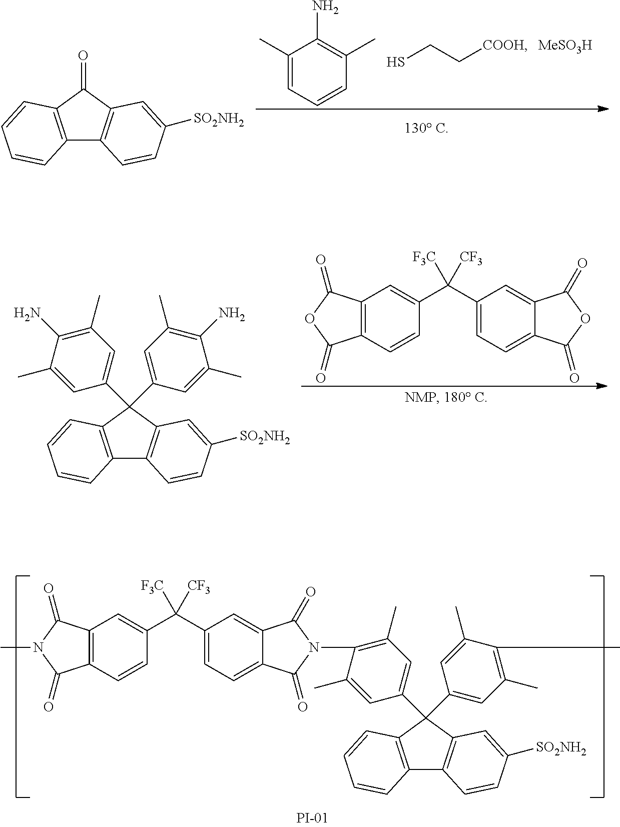

### (Synthesis of 9,9-bis(4-amino-3,5-dimethylphenyl)-9H-fluorene-2-sulfonamide)

20.74 g (80.0 mmol) of 9-fluorenone-2-sulfonamide (described in Societatis Scientiarum Lodziensis Acta Chimica, 1966, 11, 143 to 152), 48.47 g (400 mmol) of 2,6-dimethylaniline (manufactured by Tokyo Chemical Industry Co., Ltd.), 339.6 mg (3.20 mmol) of 3-mercaptopropionic acid (manufactured by Tokyo Chemical Industry Co., Ltd.), and 115.33 g (1,200 mmol) of methanesulfonic acid (manufactured by Tokyo Chemical Industry Co., Ltd.) were mixed and stirred at 130° C. for 8 hours. The reaction solution was cooled to room temperature, added to a 1 L aqueous solution containing 126.0 g (1,500 mol) of sodium bicarbonate, and stirred at room temperature for 30 minutes. The deposited solid content was filtered, washed with pure water, and purified by silica gel column chromatography (developing solvent: hexane/ethyl acetate=10/90). The obtained solid was heated and completely dissolved in tetrahydrofuran (THF) and re-precipitated using hexane, thereby obtaining 8.8 g of 9,9-bis(4-amino-3,5-dimethylphenyl)-9H-fluorene-2-sulfonamide (yield of 22.7%).

NMR (400 MHz, DMSO-d₆): δ=8.03 (d, 1H), 7.93 (d, 1H), 7.83-7.79 (m, 2H), 7.42-7.33 (m, 5H), 4.46 (s, 4H), 1.95 (s, 12H) ppm.

(Synthesis of Polyimide PI-01)

50 g of N-methylpyrrolidone (manufactured by Wako Pure Chemical Industries, Ltd.), 7.10 g (16.0 mmol) of a 4,4′-(hexafluoroisopropylidene)diphthalic anhydride (manufactured by Tokyo Chemical Industry Co., Ltd.), and 7.74 g (16.0 mmol) of 9,9-bis(4-amino-3,5-dimethylphenyl)-9H-fluorene-2-sulfonamide were mixed and stirred at 180° C. for 8 hours. The reaction solution was cooled to room temperature and diluted with 25 mL of acetone (manufactured by Wako Pure Chemical Industries, Ltd.). Thereafter, 400 mL of methanol (manufactured by Wako Pure Chemical Industries, Ltd.) was added to the mixed solution to carry out re-precipitation while the mixed solution was fully stirred, and the resultant was filtered and washed with methanol. The operation in which the obtained powder was dispersed in 400 mL of methanol, filtered, and washed with methanol was repeatedly performed four times, thereby obtaining polyimide PI-01 (13.7 g, yield of 96.0%, number average molecular weight (Mn): 29,000, weight-average molecular weight (Mw): 124,000).

<Synthesis of Polyimides PI-02 to PI-08>

Polyimides PI-02 to PI-08 respectively having a structure formed of the following repeating unit were synthesized in the same manner as in the synthesis of the polyimide PI-01. In the polyimide PI-07, the number provided for each repeating unit indicates the molar ratio.

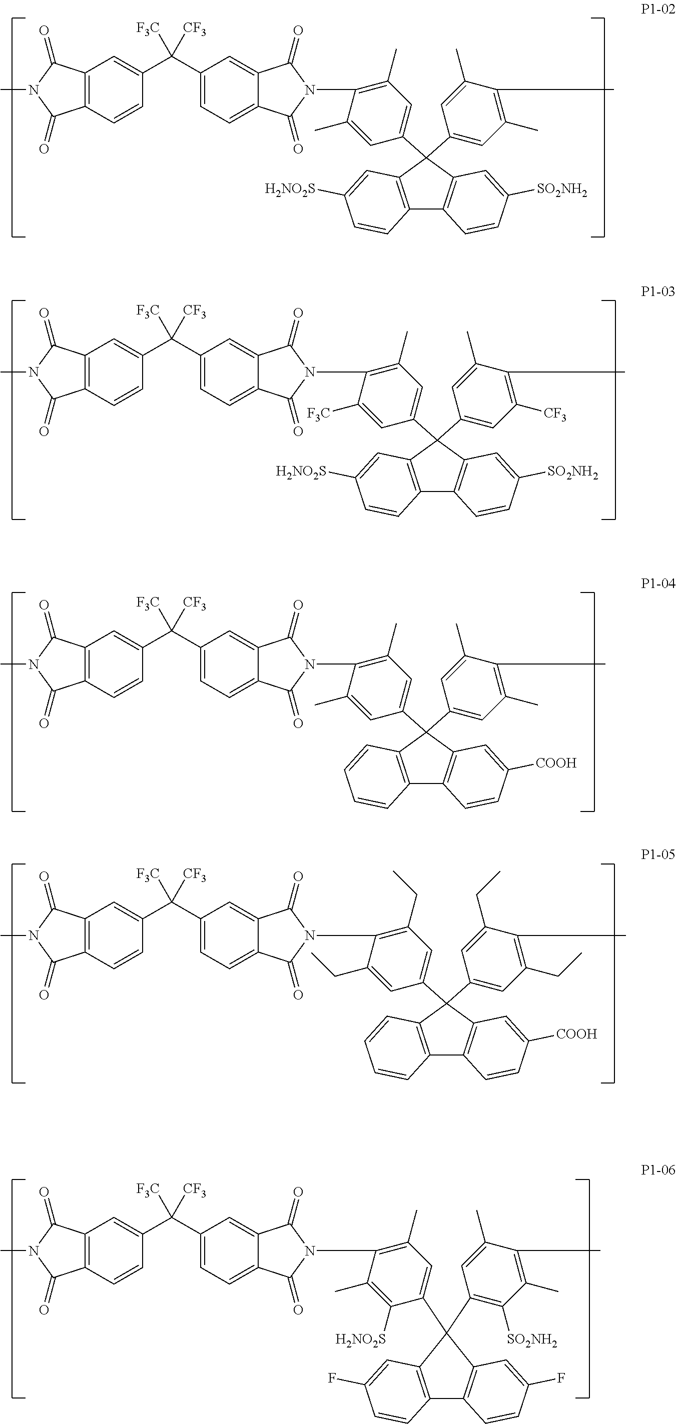

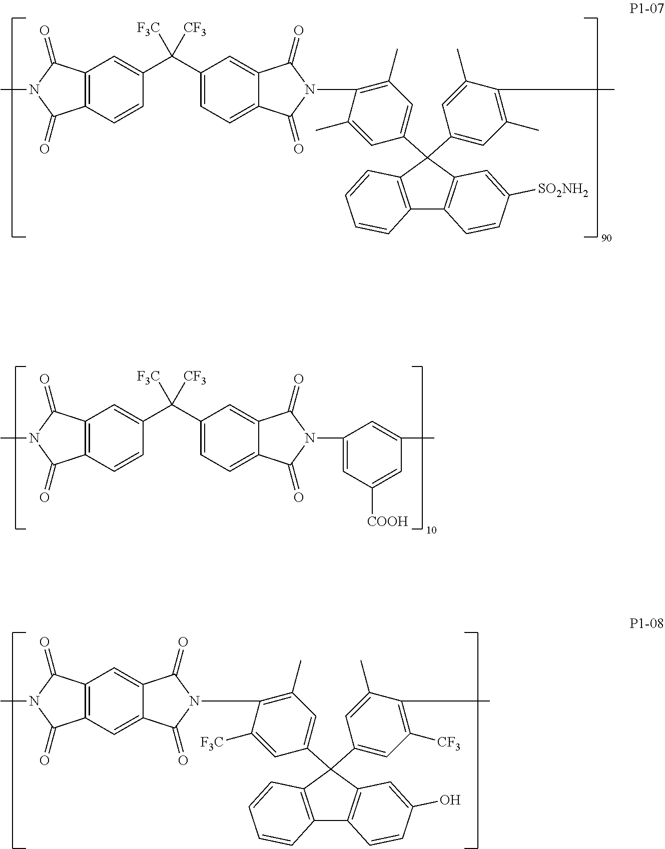

The Mn and Mw of each of the polyimides PI-01 to PI-08 are collectively listed in Table 1.

TABLE 1

| | Mn | Mw |
|---|---|---|
| PI-01 | 29000 | 124000 |
| PI-02 | 27000 | 115000 |
| PI-03 | 24000 | 57000 |
| PI-04 | 31000 | 69000 |
| PI-05 | 79000 | 216000 |
| PI-06 | 21000 | 55000 |
| PI-07 | 32000 | 137000 |
| PI-08 | 23000 | 62000 |

<Synthesis of Comparative Polyimides C-1 to C-4>

Comparative polyimides C-1 to C-4 respectively formed of the following repeating unit were synthesized. The comparative polyimide C-1 is a polyimide compound described in JP2010-189578A, the comparative polyimide C-2 is a polyimide compound described in Journal of Polymer Science Part A, Polymer Chemistry, 2008, p. 4469 to 4478, the comparative polyimide C-3 is a polyimide compound described in JP1993-192552A (JP-H05-192552A), and the comparative polyimide C-4 is a polyimide compound described in JP1990-261524A (JP-H02-261524A). Further, the number provided for each repeating unit of the comparative polyimide C-2 indicates the molar ratio.

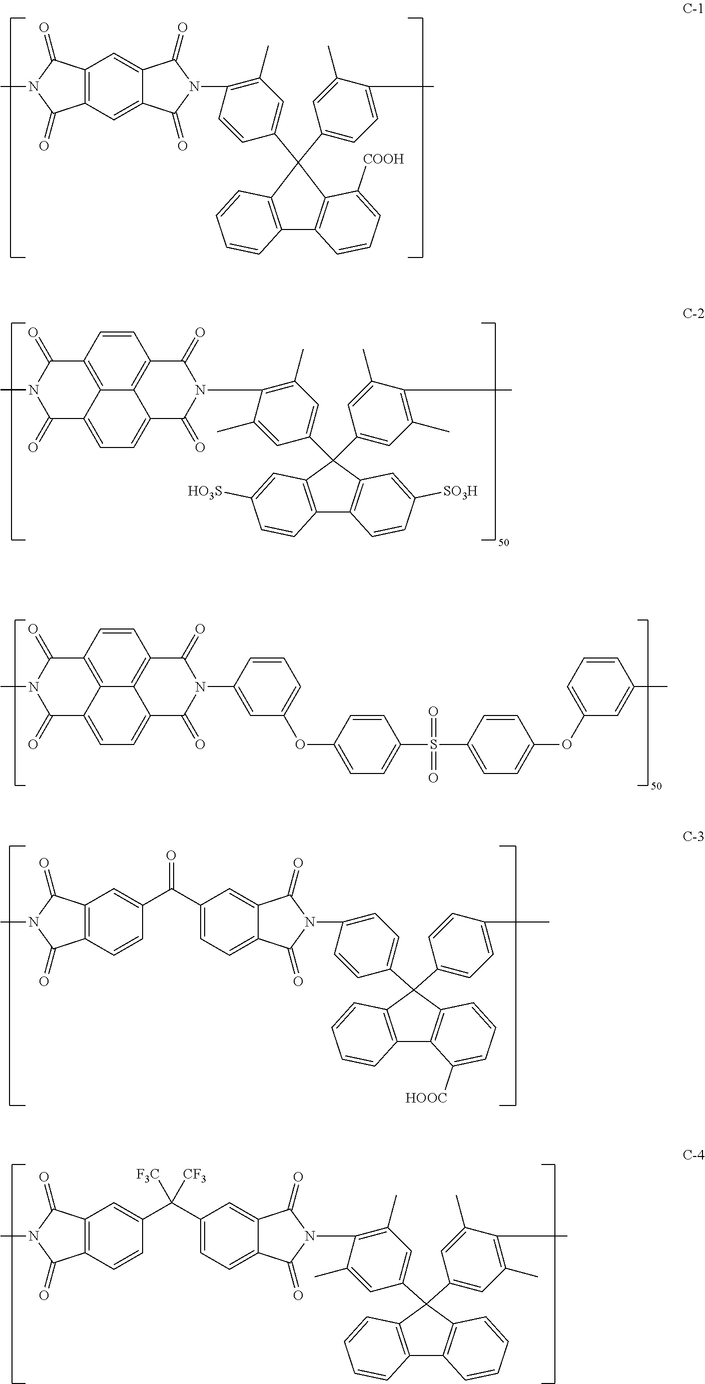

### [Example 1] Preparation of Composite Membrane

<Preparation of PAN Porous Support Provided with Smooth Layer>

(Preparation of Radiation-Curable Polymer Containing Dialkylsiloxane Group)

39 g of UV9300 (manufactured by Momentive Performance Materials Inc.), 10 g of X-22-162C (manufactured by Shin-Etsu Chemical Co, Ltd.), and 0.007 g of DBU (1,8-diazabicyclo[5.4.0]undeca-7-ene) were added to a 150 mL three-neck flask and dissolved in 50 g of n-heptane. The state of this mixed solution was maintained at 95° for 168 hours, thereby obtaining a radiation-curable polymer solution (viscosity at 25° C. was 22.8 mPa·s) containing a poly(siloxane) group.

(Preparation of Polymerizable Radiation-Curable Composition)

5 g of the obtained radiation-curable polymer solution was cooled to 20° C. and diluted with 95 g of n-heptane. 0.5 g of UV9380C (manufactured by Momentive Performance Materials Inc.) serving as a photopolymerization initiator and 0.1 g of ORGATIX TA-10 (manufactured by Matsumoto Fine Chemical Co., Ltd.) were added to the obtained solution, thereby preparing a polymerizable radiation-curable composition.

(Coating of Porous Support with Polymerizable Radiation-Curable Composition and Formation of Smooth Layer)

A porous support having a polyacrylonitrile (PAN) porous membrane present on non-woven fabric was used. The porous support was spin-coated with the polymerizable radiation-curable composition, subjected to a UV treatment (Light Hammer 10, D-valve, manufactured by Fusion UV System, Inc.) under UV treatment conditions of a UV intensity of 24 kW/m for a treatment time of 10 seconds, and then dried. In this manner, a smooth layer containing a dialkylsiloxane group and having a thickness of 1 μm was formed on the PAN porous support.

<Preparation of Composite Membrane>

A gas separation composite membrane illustrated in FIG. 2 was prepared (a smooth layer is not illustrated in FIG. 2).

0.08 g of the polyimide PI-01 and 7.92 g of tetrahydrofuran were mixed in a 30 ml brown vial bottle and then stirred for 30 minutes. The PAN porous support provided with the smooth layer was spin-coated with this mixed solution to form a gas separation layer, thereby obtaining a composite membrane. The thickness of the gas separation layer containing the polyimide (PI-01) was approximately 90 nm, and the thickness of the PAN porous support including the non-woven fabric was approximately 180 μm.

Further, as the PAN porous support, a support having a molecular weight cutoff of 100,000 or less was used. Further, the permeability of carbon dioxide from the mixed gas of Test Example 1 into this porous support under conditions of 40° C. at 5 MPa was 25,000 GPU.

### [Examples 2 to 8] Preparation of Composite Membranes

Composite membranes were prepared in the same manner as in Example 1 except that the polyimides PI-02 to PI-08 were used in place of the polyimide PI-01.

### [Comparative Examples 1 to 4] Preparation of Composite Membranes

Composite membranes of Comparative Examples 1 to 4 were prepared in the same manner as in Example 1 except that the polyimide (P-01) was changed to the comparative polymers (C-1) to (C-4).

### [Test Example 1] Evaluation of CO₂ Permeation Rate and Gas Separation Selectivity of Gas Separation Membrane

The gas separation performance was evaluated in the following manner using the gas separation membranes (composite membranes) of each of the examples and comparative examples.

Permeation test samples were prepared by cutting the gas separation membranes together with the porous supports (support layers) such that the diameter of each membrane became 5 cm. Using a gas permeability measurement device manufactured by GTR Tec Corporation, a mixed gas in which the volume ratio of carbon dioxide (CO₂) to methane (CH₄) was 13:87 was adjusted and supplied such that the total pressure on the gas supply side became 5 MPa (partial pressure of CO₂: 0.65 MPa), the flow rate thereof became 500 mL/min, and the temperature thereof became 40° C. The permeating gas was analyzed using gas chromatography. The gas permeabilities of the gas separation membranes were compared to each other by calculating gas permeation rates as gas permeability (Permeance). The unit of gas permeability (gas permeation rate) was expressed by the unit of GPU [1 GPU=1×10⁻⁶ cm³ (STP)/cm²·sec·cmHg]. The gas separation selectivity was calculated as the ratio (R_{CO2}/R_{CH4}) of the permeation rate R_{CH4} of CH₄ to the permeation rate R_{CO2} of CO₂ of the membrane.

### [Test Example 2] Evaluation of Plasticity Resistance

Each gas separation membrane of the examples and the comparative examples, used in Test Example 1, was put into a stainless steel container in which a petri dish covered with a toluene solvent was placed so that a closed system was prepared. Thereafter, each composite membrane stored under a temperature condition of 25° C. for 20 minutes was exposed to toluene vapor, and the gas separation selectivity was evaluated in the same manner as in [Test Example 1]. The ratio (A/P, the gas separation selectivity change rate after exposure to toluene vapor) of the gas separation selectivity after exposure to toluene (A) to the gas separation selectivity (P) before exposure to toluene was calculated and then used as an indicator of the plasticity resistance.

By exposing the membranes to toluene, the plasticity resistance of the gas separation membrane with respect to impurity components such as benzene, toluene, and xylene can be evaluated.

The results of each test example are listed in Table 2.

TABLE 2

| | Type of | CO₂ | | Gas separation |
|---|---|---|---|---|
| | polymer used | perme- | | selectivity change |
| | for gas sepa- | ation | R_{C02}/ | rate after exposure to |
| | ration layer | rate | R_{CH4} | toluene vapor (A/P) |
| Example 1 | PI-01 | 68 | 20 | 0.7 |
| Example 2 | PI-02 | 63 | 23 | 0.9 |
| Example 3 | PI-03 | 65 | 21 | 0.6 |
| Example 4 | PI-04 | 93 | 17 | 0.5 |
| Example 5 | PI-05 | 97 | 16 | 0.5 |
| Example 6 | PI-06 | 61 | 22 | 0.6 |
| Example 7 | PI-07 | 65 | 21 | 0.6 |
| Example 8 | PI-08 | 95 | 16 | 0.5 |
| Comparative | C-1 | 47 | 11 | 0.3 |
| Example 1 | | | | |
| Comparative | C-2 | 28 | 9 | 0.3 |
| Example 2 | | | | |
| Comparative | C-3 | 35 | 12 | 0.3 |
| Example 3 | | | | |
| Comparative | C-4 | 60 | 10 | 0.2 |
| Example 4 | | | | |

As listed in Table 2, even in a case where the diamine component of the polyimide compound used for the gas separation layer had a 4,4′-(9-fluorenylidene)dianiline skeleton, the gas permeability of the gas separation membrane was degraded and the gas separation selectivity thereof was also degraded in a case where the skeleton did not have a substituent defined in Formula (I). Further, it was understood that the gas separation selectivity was decreased to 30% or less (A/P was 0.3 or less) after exposure to toluene vapor (Comparative Examples 1 to 4).

On the contrary, the gas separation membrane including the gas separation layer formed using the polyimide compound in Formula (I) had excellent gas permeability and excellent gas separation selectivity. Further, it was understood that excellent gas separation selectivity was able to be maintained even in a case of being exposed to toluene vapor and the plasticity resistance was also excellent (Examples 1 to 8).

From the results described above, it was understood that an excellent gas separation method, an excellent gas separation module, and a gas separator provided with this gas separation module can be provided by applying the gas separation membrane of the present invention.

### EXPLANATION OF REFERENCES

1: gas separation layer
2: porous layer
3: non-woven fabric layer
10, 20: gas separation composite membrane

What is claimed is:

1. A gas separation membrane comprising:
a gas separation layer which contains a polyimide compound,
wherein the polyimide compound has a repeating unit represented by Formula (I), 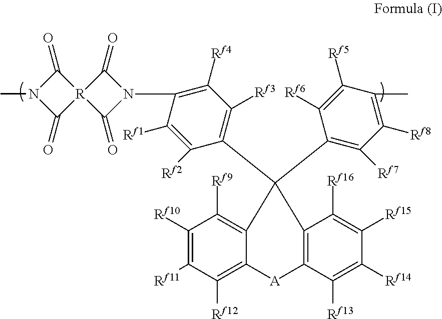
in Formula (I), A represents a divalent linking group selected from a single bond, —CR^{L1}CR^{L2}—, —O—, —S—, and —NR^{L3}—, R^{L1}, R^{L2}, and R^{L3} each independently represent a hydrogen atom or a substituent,
R^{f1}, R^{f4}, R^{f5}, and R^{f8} each independently represent an alkyl group,
R^{f2}, R^{f3}, R^{f6}, R^{f7}, and R^{f9} to R^{f16} each independently represent a hydrogen atom or a substituent,
provided that at least one of R^{f2}, R^{f3}, R^{f6}, R^{f7}, and R^{f9}, . . . , or R^{f16} represents a polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom, and
R represents a tetravalent group represented by any of Formulae (I-1) to (I-28), where X¹ to X³ each independently represent a single bond or a divalent linking group, L represents —CH═CH— or —CH₂—, R¹ and R² each independently represent a hydrogen atom or a substituent, and the symbol “*” represents a bonding site with respect to a carbonyl group in Formula (I).

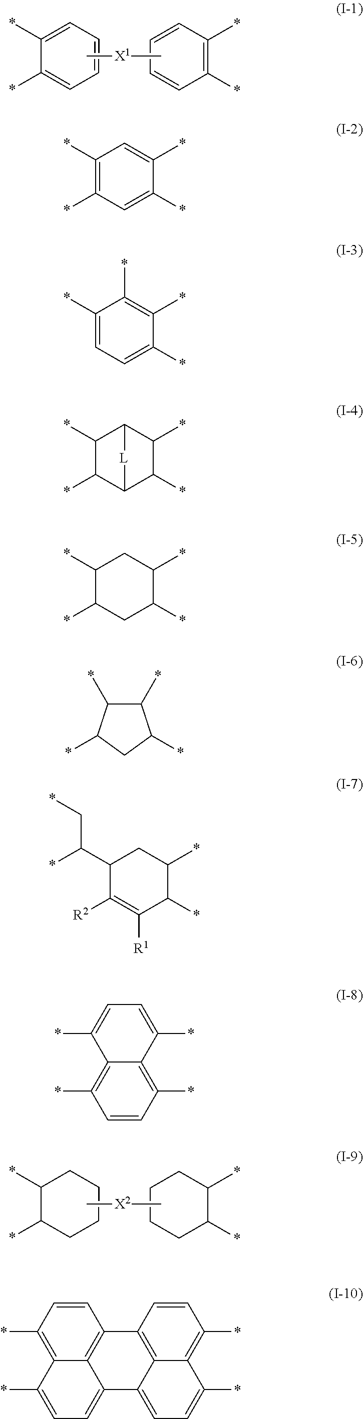

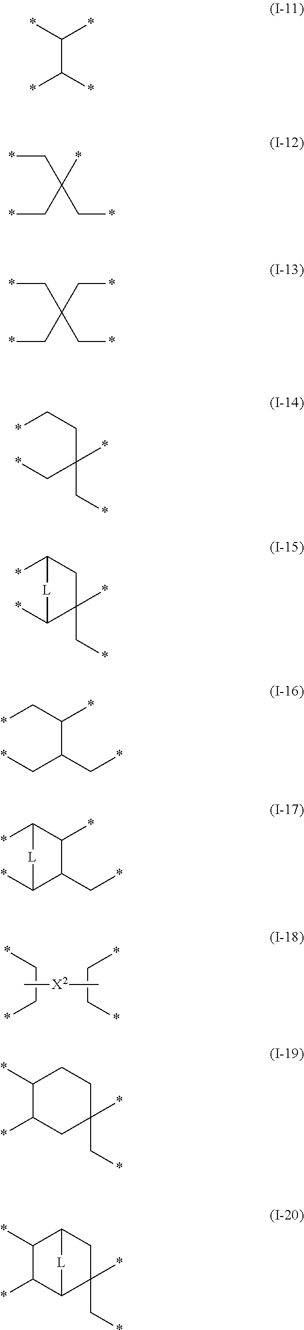

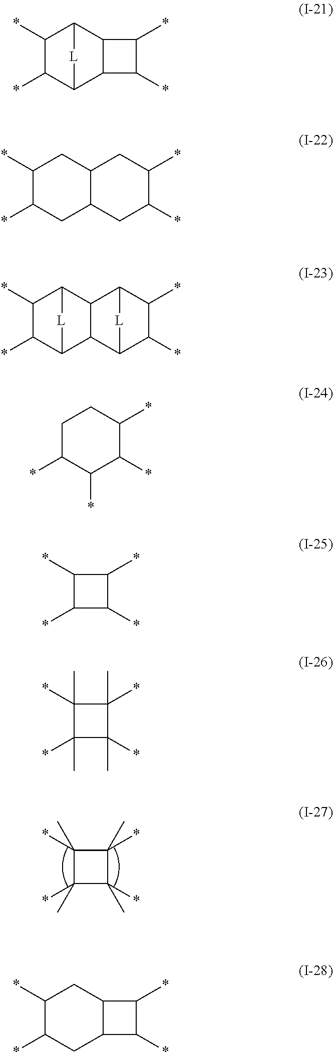

2. The gas separation membrane according to claim 1,
wherein A in Formula (I) represents a single bond.

3. The gas separation membrane according to claim 1,
wherein R^{f10} and/or R^{f15} in Formula (I) represents a polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom.

4. The gas separation membrane according to claim 1,
wherein any two to four of R^{f2}, R^{f3}, R^{f6}, R^{f7}, and R^{f9} to R^{f16} in Formula (I) represent a polar group selected from a sulfamoyl group, a carbamoyl group, a carboxy group, a hydroxy group, an acyloxy group, an alkoxycarbonyl group, an aryloxycarbonyl group, a cyano group, a nitro group, and a halogen atom.

5. The gas separation membrane according to claim 1,
wherein at least one of R^{f2}, R^{f3}, R^{f6}, R^{f7}, and R^{f9}, . . . , or R^{f16} in Formula (I) represents a sulfamoyl group.

6. The gas separation membrane according to claim 1,
wherein R^{f1}, R^{f4}, R^{f5}, and R^{f8} in Formula (I) represent methyl.

7. The gas separation membrane according to claim 1,
wherein the repeating unit represented by Formula (I) is represented by Formula (I-a), 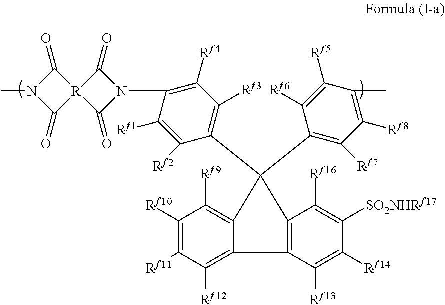
in Formula (I-a), R^{f1} to R^{f14}, R^{f16}, and R each have the same definition as that for R^{f1} to R^{f14}, R^{f16}, and R in Formula (I), and R^{f17} represents a hydrogen atom or a substituent.

8. The gas separation membrane according to claim 1,
wherein the polyimide compound further has at least one repeating unit selected from a repeating unit represented by Formula (II-a) and a repeating unit represented by Formula (II-b), 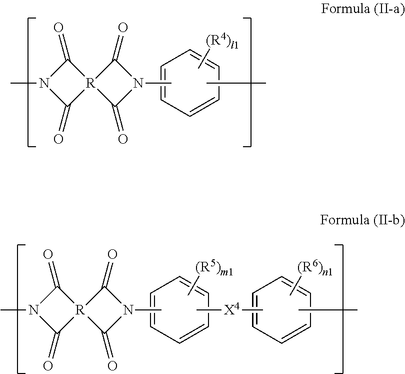
in Formulae (II-a) and (II-b), R has the same definition as that for R in Formula (I), R⁴ to R⁶ each independently represent a substituent, l1, m1, and n1 each independently represent an integer of 0 to 4, and X⁴ represents a single bond or a divalent linking group, provided that the repeating unit represented by Formula (II-b) does not include the repeating unit included in the repeating unit represented by Formula (I).

9. The gas separation membrane according to claim 8,
wherein a ratio of a molar amount of the repeating unit represented by Formula (I) to a total molar amount of the repeating unit represented by Formula (I), the repeating unit represented by Formula (II-a), and the repeating unit represented by Formula (II-b) in the polyimide compound is 50% by mole or greater and less than 100% by mole.

10. The gas separation membrane according to claim 8,
wherein the polyimide compound is formed of the repeating unit represented by Formula (I) and the repeating unit represented by Formula (II-a), the repeating unit represented by Formula (I) and the repeating unit represented by Formula (II-b), or the repeating unit represented by Formula (I), the repeating unit represented by Formula (II-a) and the repeating unit represented by Formula (II-b).

11. The gas separation membrane according to claim 1,
wherein the polyimide compound does not have any of a repeating unit represented by Formula (II-a) and a repeating unit represented by Formula (II-b), 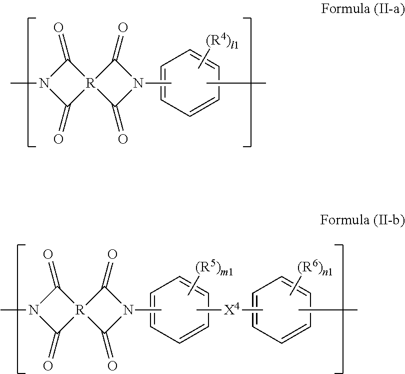
in Formulae (II-a) and (II-b), R has the same definition as that for R in Formula (I), R⁴ to R⁶ each independently represent a substituent, l1, m1, and n1 each independently represent an integer of 0 to 4, and X⁴ represents a single bond or a divalent linking group, provided that the repeating unit represented by Formula (II-b) does not include the repeating unit included in the repeating unit represented by Formula (I).

12. The gas separation membrane according to claim 11,
wherein the polyimide compound is formed of the repeating unit represented by Formula (I).

13. The gas separation membrane according to claim 1,
wherein the gas separation membrane further comprises a gas permeating support layer and is a gas separation composite membrane in which the gas separation layer is provided on the upper side of the gas permeating support layer.

14. The gas separation membrane according to claim 13,
wherein the gas permeating support layer includes a porous layer and a non-woven fabric layer, and
the gas separation layer, the porous layer, and the non-woven fabric layer are provided in this order.

15. The gas separation membrane according to claim 1,
wherein a permeation rate of carbon dioxide in a mixed gas containing carbon dioxide and methane at 40° C. and 5 MPa is greater than 20 GPU, and a ratio (R_{CO2}/R_{CH4}) between permeation rates of the carbon dioxide and the methane is 15 or greater.

16. The gas separation membrane according to claim 1, which is used for selective permeation of carbon dioxide from the mixed gas containing carbon dioxide and methane.

17. A gas separation module comprising:
the gas separation membrane according to claim 1.

18. A gas separator comprising:
the gas separation module according to claim 17.

19. A gas separation method which is performed by using the gas separation membrane according to claim 1.
